(19) United States
(12) Patent Application Publication
(10) Pub. No.: US 2008/0006536 A1
(43) Pub. Date: Jan. 10, 2008

(54) Processing cellulosic material utilizing atmospheric-pressure plasma

(75) Inventors: Jerome J. Cuomo, Cary, NC (US); Christopher J. Oldham, Raleigh, NC (US); Matthew R. King, Raleigh, NC (US)

Correspondence Address: THE ECLIPSE GROUP, 10605 BALBOA BLVD., SUITE 300, GRANADA HILLS, CA, 91344, US

(73) Assignee: North Carolina State University, Raleigh, NC (US)

(21) Appl. No.: 11/804,261

(22) Filed: May 17, 2007

Related U.S. Application Data
(60) Provisional application No. 60/801,442, filed on May 18, 2006.

(51) Int. Cl. C12P 7/06; B03C 5/02; C13K 1/02; H05H 1/03; C08B 15/00

(52) U.S. Cl. 204/672; 127/30; 127/34; 127/37; 204/157.68; 204/660; 204/671; 435/161

(57) ABSTRACT

Cellulosic material is treated by atmospheric-pressure (AP) plasma to enhance processes for extracting sugars from cellulosic material and fermenting sugars into alcohols or other chemicals. In one example, the AP plasma treatment is utilized to improve the release, activation or production of glucose and conversion of glucose to ethanol. The AP plasma treatment may be performed in conjunction with other processes such as depolymerization or degradation, for example hydrolysis, as well as fermentation. The AP plasma treatment may be performed as a substitute for pretreatment processes such as steam explosion, and in some implementations is sufficiently effective to serve as a substitute for hydrolytic processes or at least as an enhancement to hydrolytic processes or other depolymerization or degradation processes.

### RELATED APPLICATIONS

This application claims the benefit of U.S. Provisional Patent Application Ser. No. 60/801,442, filed May 18, 2006, titled “PROCESSING CELLULOSIC MATERIAL UTILIZING ATMOSPHERIC-PRESSURE PLASMA;” the content of which is incorporated by reference herein in its entirety.

### BACKGROUND OF THE INVENTION

1. Field of the Invention

This invention relates generally to the processing of biomass such as cellulosic material to produce sugars and the fermentation of such sugars to produce alcohols and other chemicals. More particularly, the invention relates to the utilization of atmospheric-pressure plasma to enhance such processing.

2. Related Art

Cellulosic materials, including lignocellulosic materials, biomass, etc., occur abundantly in nature and constitute a significant source of sugars from which alcohols and other industrial chemicals may be derived. Cellulose, hemicellulose, and lignin are three primary components of cellulosic materials. Cellulose forms the primary structural component of plant cell walls. The secondary cell wall of green plants contains lignin as well as cellulose. Lignocellulose (cellulose and lignin) such as wood is the most common terrestrial biopolymer, by some accounts comprising approximately 50% of the biomass in the world. See M. Galbe & G. Zacchi, “A review of the production of ethanol from softwood,” Appl. Microbiol. Biotechnol., Vol. 59, pp. 618-628, Springer-Verlag (2002). Cellulose holds particular interest because it can be processed to yield glucose monomers. Glucose can be converted to fuel-grade alcohols such as ethanol (CH₃CH₂OH, or C₂H₆O) by fermentation (i.e., bioethanol).

Galbe, supra, and others have investigated the merits of bioethanol as a renewable energy source. Rendering bioethanol commercially available is considered to be a viable way for reducing the environmental effects of and dependence on fossil fuels. The combustion of bioethanol results in low emissions of carbon dioxide (CO₂), as well as carbon monoxide (CO), non-combusted hydrocarbons (HCs), nitrogen oxides (NOₓ), and volatile organic compounds (VOCs). Moreover, the mixing of ethanol and gasoline is advantageous in that the higher octane number of ethanol (96-113) increases the octane number of the mixture and thereby reduces the need for toxic, octane-enhancing additives, the ethanol supplies oxygen for the fuel and thus enables cleaner combustion, and ethanol is believed to be about 15% more efficient than gasoline. Accordingly, although ethanol has only about two-thirds of the volumetric energy content of gasoline, it would still be possible to drive 75-80% of the distance on a given volume of ethanol. See Galbe, supra. For reasons such as the foregoing, cellulosic materials are considered to be an important potential renewable source—particularly a domestic source of alternative fuels—and thus the efficient conversion of cellulosic components to alcohols, particularly ethanol, is the subject of ongoing research.

Cellulosic materials exist in nature in a variety of different compositions and structures. As a general example, a typical cellulosic material may be considered as being a heterogeneous, three-dimensional composite or complex of cellulose fibers wrapped in a sheath of hemicellulose and lignin. The cellulosic material typically includes crystalline regions as well as less ordered amorphous regions. The ratios of the three primary components of the cellulosic material—cellulose, hemicellulose, and lignin—relative to each other depend on the species of the cellulose-containing material (e.g., various woods, grains, corn stover, etc.). In addition to the three primary components, the cellulosic material may include lower organic components and mineral components of lesser immediate interest for the purposes of the present disclosure.

Cellulose and hemicellulose are carbohydrate polymers. Cellulose is a long-chain polysaccharide carbohydrate of β-glucose monomers, which may be chemically represented as (C₆H₁₀O₅)ₙ. More specifically, cellulose is a polymer of D-glucose (C₆H₁₂O₆) with β [1→4] linkages (glycosidic bonds) between each of the about 500 to 10,000 glucose units. Cellulose is a straight-chain polymer that exhibits a rod-like conformation, unlike starch which exhibits coiling. Cellulose constitutes about 35-60% by weight of typical cellulosic materials. Hemicellulose is a non-cellulosic, heteropolymer polysaccharide of primarily D-xylose (C₅H₁₀O₅) and other pentoses and some hexoses with β [1→4] linkages. Hemicellulose may be found as a branched polymer of glucose or xylose, substituted with arabinose, xylose, galactose, fucose, mannose, glucose, or glucuronic acid. The molecular weights of hemicellulose polymers are usually lower than that of cellulose, and hemicellulose polymers have a weak undifferentiated structure compared to crystalline cellulose. Hemicellulose, however, binds with pectin (a heterosaccharide) to cellulose to form a network of cross-linked fibers that serves as the structural backbone of plant cell walls. Hemicellulose constitutes about 20-35% by weight of typical cellulosic materials. Lignin may be characterized as a complex, cross-linked, random, amorphous, three-dimensional polyphenolic polymer that typically is based on variously substituted p-hydroxyphenlypropane units. Lignin generally permeates the matrix of cellulose fibers and largely fills in the interstices between the cellular structures (cellulose, hemicellulose and pectin components) of the cellulosic material. However, lignin appears to be more intimately cross-linked or otherwise associated with hemicellulose than with the distinct crystalline phase of cellulose. Lignin constitutes about 10-30% by weight of typical cellulosic materials.

Cellulosic materials are converted to alcohols by releasing the component sugars of the cellulosic materials, and fermenting the sugars to alcohols. The carbohydrate polymers of cellulosic materials are typically depolymerized (degraded or broken down) into fermentable monomeric sugars by hydrolysis. Component sugars may include six-carbon sugars (hexoses) such as glucose, galactose, and mannose, and five-carbon sugars (pentoses) such as xylose and arabinose. Both chemical and enzymatic hydrolytic processes have been utilized.

Chemical hydrolysis typically entails the use of an acid such as sulfuric acid as a catalyst. Generally, microcrystalline cellulose is relatively resistant to typical acid hydrolysis, amorphous cellulose is less resistant, hemicellulose (which is also amorphous) is even less resistant, and lignin is highly resistant but may be dissolved by certain organic solvents. Acid hydrolysis utilizes either concentrated acids or diluted acids. Acid hydrolysis generally is around 10-40% efficient in terms of sugar recovery, depending on process conditions. Concentrated acid hydrolysis involves short reaction times, but requires a large amount of expensive acid(s), corrosion-resistant equipment, and energy-demanding means for recycling the acid. Moreover, concentrated acid hydrolysis requires significant control over the reaction to avoid degrading the desired sugars and forming toxic byproducts. Dilute acid hydrolysis is a lower cost process involving a relatively low consumption of acid(s), but requires longer reaction times and results in a decreased glucose yield as compared to concentrated acid hydrolysis. Moreover, dilute acid hydrolysis requires high temperatures to attain acceptable rates of conversion of cellulose to sugar monomers. High temperatures require a high input of energy, promote equipment corrosion, and increase the rates of hemicellulose-derived sugar decomposition. For example, the products of decomposing hemicellulose may include furfural and hydroxymethylfurfual. It is known that sugar decomposition products can inhibit the subsequent fermentation process.

To reduce sugar decomposition, a two-stage acid hydrolysis process has been employed. The first stage is carried out under relatively mild conditions to release sugars as a result of hydrolysis of the hemicellulose, and the second stage is carried out under relatively harsher conditions to hydrolyze the cellulose fraction. The first stage enables the second stage to proceed under the harsher conditions without decomposing the hemicellulose into undesired by-products, but the glucose yield is still unacceptably low (e.g., 50%). See Galbe, supra.

Enzymatic hydrolysis (e.g., biodegradation) may entail the use of a variety of microorganisms, which may be naturally occurring or genetically engineered. Enzymes include carbohydrases such as cellulases and hemicellulases. More than one type of enyzme may be employed, and their combined effect may be synergistic. For example, the combined action of the three cellulase enzymes endo-β-glucanase, exo-β-glucanase, and β-glucosidase (cellobiase) has been employed to convert cellulose into the glucose monomer. The endocellulase breaks internal bonds to disrupt the crystalline structure of cellulose and expose individual cellulase polysaccharide chains. The exocellulase cleaves two to four units from the ends of the exposed chains produced by the endocellulase, resulting in tetrasaccharides or disaccharide such as cellobiose. The glucosidase hydrolyzes the endocellulase product into individual monosaccharides. Similarly, mixtures of hemicellulases such as xylases have been employed to hydrolyze the xylose component of hemicellulose. Also, certain enzymes may be effective in cleaving lignin. Due to the specific activity of enzymatic catalysts, enzymatic hydrolysis has been thought to have the potential for higher monomeric sugar yields and reduced formation of toxic compounds as compared to acid hydrolysis. However, the efficiency of enzymatic hydrolysis is typically low (less than 20%), although may be improved by employing an excessive amount of enzyme. Moreover, the rates of conversion of cellulose to sugar is typically very slow due to the cellulose being protected by the matrix of hemicellulose and lignin.

Fermentation of hydrolyzate sugars involves the use of digesting or metabolizing agents such as yeast. Yeast readily metabolizes glucose, which is the predominant hydrolyzate of many types of cellulosic materials. Yeast, however, cannot metabolize other hydrolyzates such as xylose, and thus other organisms such as certain species of bacteria (e.g., Zymonmonas sp. and E. coli) have been employed for this purpose, including organisms genetically engineered to consume a specific type of hydrolyzate such as xylose. The stoichiometric expressions for the conversion of glucose and xylose into ethanol are, respectively:
C₆H₁₂O₆→2C₂H₆O+2CO₂ and
3C₅H₁₀O₅→5C₂H₆O+5CO₂.

Hydrolysis may be performed separately from fermentation in processes termed separate hydrolysis and fermentation (SHF), or may be performed simultaneously with fermentation in processes termed simultaneous saccharification and fermentation (SSF). Cellulose and hemicellulose may be fermented separately, or may be fermented together in processes termed simultaneous saccharification co-fermentation (SSCF).

Once fermentation is completed, the resulting alcohols may be separated (e.g., distilled) and purified according to any suitable processes. Residual components of the fermentation process may include lignin, unreacted cellulose and hemicellulose, ash, enzymes, microorganisms, etc.

Due to its crystalline structure, cellulose is generally water-insoluble and resistant to depolymerization. The highly packed and crystalline structure of cellulose also means that the surface area available for hydrolytic and fermentative activity is low. Moreover, as noted above, the presence of hemicellulose and lignin impedes hydrolysis of the cellulose. Hemicellulose hydrogen-bonds to cellulose to form the afore-mentioned cross-linked network. Lignin, as a large and complex macromolecule, is difficult to degrade, which renders it an effective physical barrier to plant pathogens and pests but at the same time a detrimental protection against the desired depolymerization of cellulose. Moreover, in the case of enzymatic hydrolysis, lignin is thought to bind to cellulase and thereby interfere with its ability to digest cellulose. As a result, the efficiency and costs associated with the conversion of cellulosic material into alcohols are less than desirable. In view of this, various pre-treatment methods have been proposed that endeavor to disrupt the cellulose-hemicellulose-lignin complex, expose the cellulose, and/or modify the pores of the matrix, and thereby make the cellulose more available for hydrolysis such as by allowing enzymes to penetrate into the fibers of the matrix. Pre-treatment methods have included comminution (e.g., milling, chopping, etc.), uncatalyzed steam explosion, catalyzed steam explosion (e.g., using H₂SO₄ or SO₂), hydrothermolysis (the addition of liquid hot water), the addition of acids (e.g., H₂SO₄, HCl), bases or alkalis (e.g., NaOH, lime), solvents (e.g., organosolv, ethylene glycol) and ammonia, wet oxidation (e.g., treatment of biomass with water and air or oxygen at temperatures above 120° C., sometimes also adding an alkali catalyst), ammonia fiber/freeze explosion (AFEX), ammonia recycled percolation (ARP), and other known techniques. Pre-treatment methods such as these have been summarized in Mosier et al., “Features of promising technologies for pretreatment of lignocellulosic biomass,” Bioresource Technology, Vol. 96, pp. 673-686, Elsevier (2005); M. Galbe & G. Zacchi, “A review of the production of ethanol from softwood,” Appl. Microbiol. Biotechnol., Vol. 59, pp. 618-628, Springer-Verlag (2002); as well as in other literature. Many of these pre-treatment methods require high pressures, temperatures and energy inputs, and are costly.

Therefore, in view of the foregoing, despite some advances in pre-treatment and hydrolytic methods, it is well-recognized by persons skilled in the art that an ongoing need exists for providing improved methods, apparatus and systems for the conversion of cellulosic materials to sugars and further conversion of sugars into alcohols and other chemicals. Higher efficiency is desired in terms of yield, energy consumption, and cost.

### SUMMARY

According to one implementation, a method is provided for treating a cellulosic material. The method includes subjecting the cellulosic material to an atmospheric-pressure plasma.

According to another implementation, a method is provided for treating a cellulosic material. According to the method, the cellulosic material is subjected to an atmospheric-pressure plasma to produce a plasma-treated cellulosic material. One or more components of the plasma-treated cellulosic material are subjected to a degradation process. Examples of the degradation process include, but are not limited to, various acid hydrolysis and enzymatic hydrolysis processes.

According to another implementation, a method is provided for treating a cellulosic material. According to the method, the cellulosic material is subjected to a first degradation process to produce a first degradation-processed cellulosic material. One or more components of the first degradation-processed cellulosic material are subjected to an atmospheric-pressure plasma to produce a plasma-treated cellulosic material. One or more components of the plasma-treated cellulosic material are subjected to a second degradation process. Examples of the first and second degradation processes include, but are not limited to, various acid hydrolysis and enzymatic hydrolysis processes.

According to another implementation, a method is provided for treating a cellulosic material. According to the method, the cellulosic material is subjected to a pretreatment process, an atmospheric-pressure plasma treatment, and a degradation process. Examples of the pretreatment process include, but are not limited to, comminution, steam explosion, hydrothermolysis, the addition of acids, bases, solvents, or ammonia, wet oxidation, ammonia fiber/freeze explosion (AFEX), ammonia recycled percolation (ARP), hydrolysis, and combinations of two or more of the foregoing.

According to implementations disclosed herein, the atmospheric-pressure plasma treatments and/or the degradation processes may produce sugars. According to some implementations, one or more of these sugars may be further processed as needed to produce sugars of commercial-grade quality. According to other implementations, one or more of these sugars may be subjected to one or more fermentation processes as desired to produce one or more types of alcohols or other chemicals.

According to implementations disclosed herein, atmospheric-pressure plasma treatment may include introducing the cellulosic material to a plasma-generating apparatus, and operating the apparatus to generate the atmospheric-pressure plasma from a plasma medium provided to the apparatus. In some implementations, the atmospheric-pressure plasma treatment may include operating a dielectric barrier discharge apparatus. In some implementations, the atmospheric-pressure plasma treatment may include operating a plasma-generating apparatus configured as, for example, a parallel-plate reactor, a drop-tube reactor, a fluidized-bed reactor, or a liquid-bath reactor, a plasma jet apparatus, or a microplasma-generating apparatus.

According to other implementations, devices, apparatus, structures, systems and/or materials are provided for practicing methods or processes disclosed herein.

According to another implementation, a sugar such as, for example, glucose is provided that is produced according to one or more implementations disclosed herein.

According to another implementation, a fermentation product is provided that is produced according to one or more implementations disclosed herein. Examples of fermentation products include, but are not limited to, organic compounds such as, for example, alcohols, examples of which include ethanol among other compounds.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 is a flow diagram illustrating an example of a method for treating cellulosic material according to one or more implementations of the invention.

FIG. 2 is a flow diagram illustrating an example of another method for treating cellulosic material according to one or more other implementations of the invention.

FIG. 3 is a flow diagram illustrating an example of another method for treating cellulosic material according to one or more other implementations of the invention.

FIG. 4 is a flow diagram illustrating an example of another method for treating cellulosic material according to one or more other implementations of the invention.

FIG. 5 is a schematic diagram illustrating an example of an atmospheric-pressure (AP) plasma apparatus provided in accordance with one or more implementations of the invention.

FIG. 6 is a schematic diagram illustrating an example of another AP plasma apparatus provided in accordance with one or more other implementations of the invention.

FIG. 7 is a schematic diagram illustrating an example of another AP plasma apparatus provided in accordance with one or more other implementations of the invention.

FIG. 8 is a schematic diagram illustrating an example of another AP plasma apparatus provided in accordance with one or more other implementations of the invention.

FIG. 9 is a schematic diagram illustrating an example of another AP plasma apparatus provided in accordance with one or more other implementations of the invention.

FIG. 10 is a flow diagram illustrating an experimental procedure performed in conjunction with studying the effects of AP plasma treatment on cellulosic material.

FIG. 11 is a bar graph illustrating data acquired as a result of performing the experimental procedure illustrated in FIG. 9.

FIG. 12 is a top plan view of container employed to hold samples of cellulosic material in conjunction with another experimental procedure performed to study the effects of AP plasma treatment on cellulosic material.

### DETAILED DESCRIPTION

As used herein, the term “cellulosic material” generally encompasses any cellulose-containing material, including lignocellulosic material and biomass, either living or existing as a waste product of industry or nature. Examples of cellulosic material include, but are not limited to, the following: forestry products, including forestry wastes, such as woods of various species of trees, including softwoods (e.g., gymnosperms such as conifers, pine, spruce, etc.), hardwoods (e.g., angiosperms such as maple, poplar, etc.), etc., including in the form of log slash, bark, trunks, stumps, branches, twigs, and the like, as well as grasses (e.g., angiosperms); agricultural products, including agricultural wastes, such as corn stover, corn cobs, rice straw, orchard and vineyard trimmings, manure, etc.; biomass crops such as grasses (e.g., switch grass), woods (e.g., poplar trees), crop residue, etc.; municipal/industrial wastes such as paper, gardening and yard refuse, etc.; and the outer coverings of various fruits, vegatables, seeds, and other plant matter such as maize (e.g., corn), straw, cereals, rye, rape-seed, cotton-seed, sunflower seed, alfalfa, oats, barleys, wheat, millet, sorghum, buckwheat, bagasse, etc.; sawdust, wood chips, wooden plant material, non-wooden plant material, etc. Generally, wooden and non-wooden plant material may be in any form, including, but not limited to, stems, stalks, shrubs, foliage, leaves, bark, roots, shells, rinds, pods, nuts, husks, hulls, fibers, vines, straws, hay, grasses, bamboo, reeds, etc. Wooden material may include heartwood (e.g., duramen) as well as outer wood (e.g., xylem). Moreover, the cellulosic material may be a mixture or combination of one or more of the foregoing items.

As used herein, the term “degradation” generally encompasses any process that results in a molecule being broken down into simpler molecules, radicals, and/or charged species. For example, the term “degradation” may encompass the breaking down of a polymer into smaller polymers (e.g., oligomers, trimers, dimers, etc.) and/or monomers such as, for example, the breaking down of a cellulose into glucose units. The term “degradation” may also encompass the breaking up or removal of physical and/or chemical bonds among different types of components of a complex material, and/or bonds internal to such components. For example, degradation may encompass the breaking up of bonds between cellulose, hemicellulose, and/or lignin, and/or the breaking down of polymeric cellulose or hemicellulose into component sugars. The term “degradation” may also encompass the removal, in whole or in part, of a component from a complex material. For example, in a complex of cellulose, hemicellulose and lignin, degradation may encompass the removal of at least some of the lignin from the complex, thereby providing greater access to the cellulose and hemicellulose components. The term “degradation” may also encompass the alteration or modification of the structure of a biomaterial. For example, degradation may encompass the opening up of interstices, voids, recesses or pores (more generally, spatial features) existing within the structure of a complex of cellulose, hemicellulose and lignin, and/or the creation of new interstices, voids, recesses or pores in such material. Degradation may entail physical, chemical, and/or biological work. Degradation may entail processes that are aided or unaided by catalytic activity. In view of the foregoing, the term “degradation” encompasses such terms as depolymerization, hydrolysis, dissociation, dissolution, disruption, delignification, removal of material, conversion of a complex material into simpler components, and release or extraction of components from a complex material.

In general, the term “communicate” (for example, a first component “communicates with” or “is in communication with” a second component) is utilized in the present disclosure to indicate a structural, functional, mechanical, electrical, optical, magnetic, ionic or fluidic relationship between two or more components (or elements, features, or the like). As such, the fact that one component is said to communicate with a second component is not intended to exclude the possibility that additional components may be present between, and/or operatively associated or engaged with, the first and second components.

According to implementations of the invention, described by way of example in the present disclosure, cellulosic material or other biomass is treated by atmospheric-pressure (AP) plasma. In some implementations, the treatment by AP plasma is employed as a substitute for conventionally known degradation or depolymerization processes such as acid hydrolysis and enzymatic hydrolysis. That is, the treatment by AP plasma is in some implementations sufficient to activate, expose, and/or even release or produce fermentable sugars from the cellulosic material. In other implementations, the treatment by atmospheric plasma is utilized to improve or enhance other processes for converting the cellulosic material to sugars (e.g., hydrolysis), including processes for converting the cellulosic material to fermentable sugars followed by converting the sugars to alcohols as well as other industrial chemicals. As compared to conventional hydrolytic techniques, AP plasma treatment is less harsh or rigorous in terms of its effects on cellulosic material and the process conditions required. Accordingly, the AP plasma treatment of cellulosic materials may be characterized as a “soft” degradation technique (e.g., soft depolymerization, soft hydrolysis, etc.). The treatment by AP plasma renders the cellulosic material more susceptible or accessible to methods for breaking down the cellulosic material into constituent sugars—such as glucose in the case of cellulose, and xylose and/or other pentoses in the case of most hemicelluloses—such methods including chemical hydrolysis and enzymatic hydrolysis. Moreover, the treatment by AP plasma renders fermentation techniques for producing chemicals of interest (e.g., ethanol) more effective, including techniques entailing co-fermentation of more than one type of sugar and techniques entailing simultaneous depolymerization and fermentation. Accordingly, the treatment by AP plasma facilitates not only the extraction of sugars but also the conversion of the hydrolyzate sugars into ethanol and/or other alcohols and chemicals of interest. As such, the treatment by AP plasma is a low-cost, low-energy (e.g., low-temperature, low electrical demand) alternative to conventional treatments. Moreover, in at least some implementations, AP plasma treatment enables the conversion of cellulosic material to sugars or further to alcohols or other chemicals to be performed as a continuous process.

The structure and composition of typical cellulosic materials is briefly summarized above and described in more detail in readily available literature. Without wishing to be bound by any particular theory, it is believed that the application of AP plasma in accordance with the invention degrades the coating (e.g., lignin) protecting the cellulose and opens up the cellulose-hemicellulose-lignin complex by enlarging spatial features existing in the complex and/or creating new spatial features, thereby creating greater access to internal structures of value, i.e., saccharide components. It is also believed that the AP plasma treatment prevents further interference from secondary protective coatings such as lignin and other binders in biomass material. Additionally, or alternatively, it is believed that the AP plasma disrupts at least some of the bonds or linkages existing within the complex, including bonds between the cellulose, hemicellulose and lignin (e.g., delignification) and/or at least some of the bonds or linkages existing within one or more individual components of the cellulose, hemicellulose and lignin. As a result, the treatment by AP plasma renders the cellulose component of the cellulosic material more amenable to hydrolytic cleavage or other types of depolymerization and, more generally, increases both the chemical and biochemical reactivity of the cellulose. The AP plasma-treated cellulosic material provides greater surface area available for hydrolyzing, solubilizing and fermenting activity, and greater access and contact with hydrolyzing, solubilizing and fermenting agents, thereby improving the efficiency of yield as well as the effectiveness and rates of reaction.

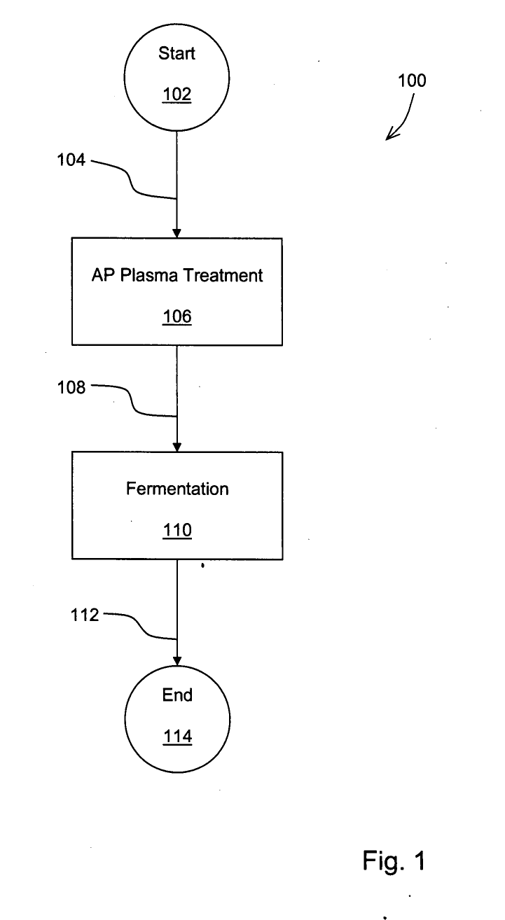

FIGS. 1-4 illustrate examples of methods for treating cellulosic material. In some implementations, such methods may be practiced for at least partially converting the cellulosic materials into sugars. In other implementations, such methods may be practiced for at least partially converting the sugars into alcohols, such as ethanol, or other desired chemicals.

FIG. 1 is a flow diagram 100 illustrating an example of a method for treating cellulosic material. Depending on the cellulosic material being processed and the process conditions, the treatment of the cellulosic material by AP plasma according to the method may, in and of itself, result in degradation of at least some of the biopolymeric components of the cellulosic material such that monomeric sugars are released (or oligomeric compounds readily convertible to monomeric sugars). In such implementations, the AP plasma treatment may be performed in conjunction with enzymatic treatment for producing alcohols or other desired chemicals, or may be directly followed by such enzymatic treatment. In other implementations, the AP plasma treatment at least may result in conditioning the cellulosic material in a manner that optimizes or facilitates a subsequent degradation process such as, for example, hydrolysis. The flow diagram 100 may also represent an apparatus or system capable of performing the illustrated method.

The method begins at starting point 102. In implementations where the cellulosic material is initially provided as a raw feedstock, the starting point 102 may be representative of any suitable preliminary steps that may be taken to prepare the cellulosic material for treatment by AP plasma. For instance, if the cellulosic material is initially provided in the form of large pieces of wood, the wood may be further comminuted into wood chips or sawdust. As another example, the cellulosic material may be washed to remove dirt or other undesired substances. As another example, the cellulosic material may be dried by any suitable means to remove moisture if desired. The cellulosic material 104 (raw feedstock, or feedstock prepared such as by the afore-mentioned preliminary steps) is introduced to an apparatus for generating an AP plasma (AP plasma apparatus). The AP plasma apparatus may be adapted for either batch processing or continuous processing, and therefore the term “introduced” is used to indicate any manner by which the cellulosic material 104 is exposed to the AP plasma such as, for example, loading or feeding the cellulosic material 104 into the AP plasma, apparatus directing an AP plasma plume or jet toward the cellulosic material, etc. At block 106, the AP plasma apparatus is operated to generate and maintain a suitable AP plasma, thereby subjecting the cellulosic material 104 to the AP plasma.

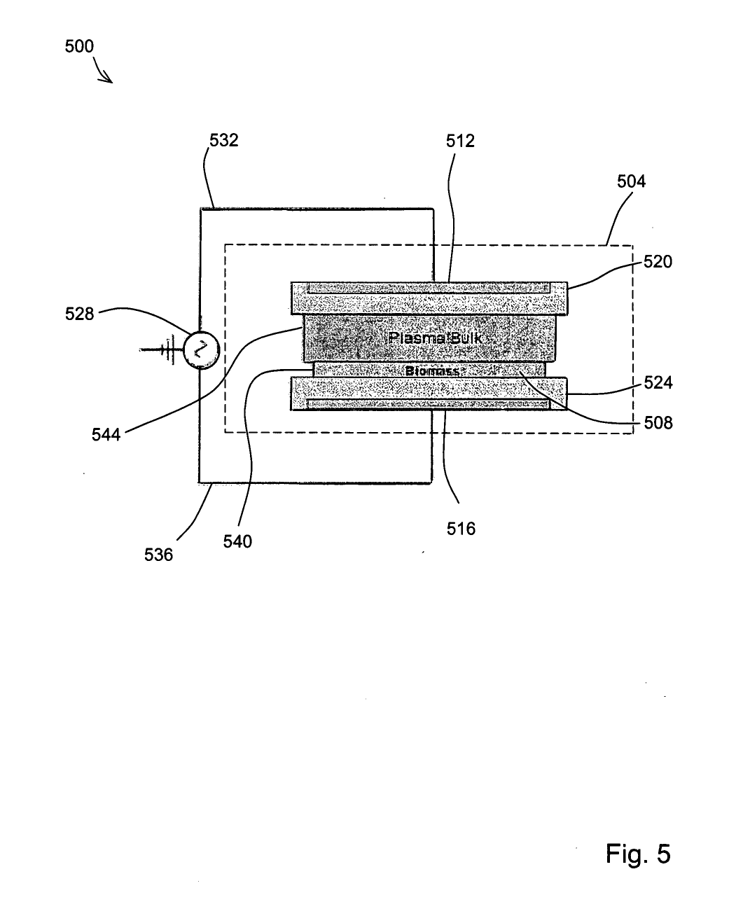

Examples of AP plasma apparatus or systems are described below with reference to FIGS. 5-9. Generally, the plasma utilized in accordance with the invention may be characterized as an ionized gas stream or cloud that may be generated in, for example, a radio frequency (RF), direct current (DC), pulsed DC, asymmetrical pulsed, or alternating current (AC) electromagnetic field, or by microwave energy. The input current for the generation of a suitable plasma may typically range from about 30 to about 300 mA, although the invention is not limited to this range. The voltage applied to the plasma may typically range from about 500 to about 50,000 V, although the invention is not limited to this range. The working frequency of the plasma may typically range from about 0.050 to about 150 kHz, although the invention is not limited to this range. The power density of the plasma may typically range from about 0.1 to about 500 W/cm³, although the invention is not limited to this range. Any suitable working gases for the AP plasma may be utilized. Examples of working gases include, but are not limited to, air, oxygen, hydrogen, helium, water-saturated helium, neon, argon, hydrogen, nitrogen, xenon, carbon dioxide, SF₆, CF₄, NH₃, and combinations of two or more of the foregoing. Flow rates may typically range from about 100 to about 50,000 standard cubic centimeters per minute (sccm), although the invention is not limited to this range. The particular species of the AP plasma that serve an active role in altering the structure or chemistry of the cellulosic material to attain the beneficial effects described herein will generally depend on the type of working gases employed. Examples of active species of the plasma may include, but are not limited to, oxygen radicals, hydroxide radicals, NOx, and ozone. For purposes of the present disclosure, the term “atmospheric pressure” is not limited to the exact value of 760 Torr but may range from, for example, about 100 to about 1000 Torr. Accordingly, as used herein, the term “atmospheric” encompasses the term “near atmospheric.” The temperature in the chamber of the apparatus containing the AP plasma may range from about 25 to about 150° C., although the invention is not limited to this range. The duration of the AP plasma treatment may range from about 1 to about 30 minutes, although the invention is not limited to this range.

The treatment of the cellulosic material 104 by AP plasma at block 106 results in an AP plasma-treated cellulosic material 108. The plasma-treated cellulosic material 108 may include oligosaccharide and/or monosaccharide species released from components of the cellulosic material (cellulose and/or hemicellulose) as a result of the AP plasma treatment 106, as well as residual polysaccharide species, lignin and other components of the cellulosic material not appreciably affected by the AP plasma treatment 106. As described previously, however, the AP plasma treatment may have the effect of removing lignin or at least disrupting the structure of lignin and its bonds so as to reduce interference of the lignin with the treatment of the cellulose. The released monosaccharide species may include hexose sugars such as, for example, glucose, galactose and/or mannose, and/or pentose sugars such as, for example, xylose and/or arabinose, and/or other monosaccharides.

At this stage, if desired, the sugars (particularly the monosaccharides) may be recovered and separated from the plasma-treated cellulosic material 108 by any suitable means such as, for example, cyclone separation, centrifugation, decanting, filtration, washing, etc. If desired, the sugars may then be subjected to any suitable purification and/or refinement processes as necessary to provide commercial-grade sugars. In the case where sugars are the intended end product, the method ends at 114.

Generally, the sugars (particularly the monosaccharides) produced or released as a result of the AP plasma treatment 106 are microbially fermentable and hence may be utilized as a fermentation medium to produce desired alcohols and/or any other desired chemicals or organic compounds such as various ketones and organic acids. Accordingly, in other implementations, as illustrated in FIG. 1, the process may continue by subjecting AP plasma treatment-derived sugars to any suitable fermentation processing 110 to produce a fermentation product 112 that includes alcohols such as ethanol or other chemicals.

Generally, the fermentation process 110 may entail the use of any microorganisms capable of converting the sugars (e.g., oligosaccharides, monosaccharides, and the like) into the desired alcohols or other chemicals. As examples, the fermenting microorganisms may be mesophilic (which grow optimally at a temperature in the range of about 20-40° C.) or thermophilic (which grow optimally at an elevated temperature above about 50° C.). The fermenting microorganisms may be naturally occurring or alternatively may be genetically engineered to effect a desired fermentation pathway. Generally, in the case of producing ethanol, any suitable ethanologenic strains of microorganisms may be employed. As an example, to convert glucose to ethanol, suitable fermenting microorganisms include yeast species such as baker's yeast, a further non-limiting example of which is Saccharomyces cerevisiae. As another example, Zymomonas mobilis may be employed to ferment glucose to ethanol. As compared to S. cerevisiae, Z. mobilis has been thought to produce higher yields of ethanol but is less robust. As another example, to convert xylose to ethanol, suitable fermenting microorganisms include Thermoanaerobacter species (e.g., T. mathranii), Zymomonas species (e.g., Z. mobilis), and certain yeast species (e.g., Pichia). It may be possible to genetically engineer microorganisms such as S. cerevisiae, Z. mobilis, and the bacteria Eschericihia coli to improve fermenting performance. As appreciated by persons skilled in the art, more than one fermentation step may be required, depending on the desired chemical(s) to be produced (e.g., ethanol, xylitol, etc.), the type(s) of sugars to be fermented (e.g., glucose, xylose, etc.), and other factors. Moreover, different fermentation processes may be carried out in the same reaction vessel or in different reaction vessels. Furthermore, fermentation may be carried out as a batch process or as a continuous process. Still further, the fermentation of different types of components of the plasma-treated cellulosic material 108 may be carried out sequentially or simultaneously.

It will be understood that the fermentation process 110 may be preceded by any suitable pre-conditioning steps deemed necessary in preparation for fermentation, such as neutralization or other pH adjustment, removal of any components deemed to act as fermentation inhibitors, and the like. In addition, the fermentation product 112 may be subjected to any suitable post-fermentation processes as needed, such as distillation and/or adsorption to separate the desired alcohols or other chemicals from the fermentation medium and concentrate and purify the alcohols or other chemicals for commercially-acceptable uses. In addition, residual materials such as lignin may be recovered for utilization as an energy source, as appreciated by persons skilled in the art. The method ends at 114.

As noted above, FIG. 1 may also represent an example of an apparatus or system 100 for treating cellulosic material. Accordingly, block 106 may be considered as depicting a means for subjecting cellulosic material to AP plasma. An example of such means is a plasma-generating apparatus or system and associated components and materials required for its operation. Specific examples of plasma-generating apparatus or systems are described elsewhere in this disclosure and are illustrated in FIGS. 5-9. Block 110 may be considered as depicting a means for fermenting plasma-treated cellulosic material. An example of such means is one or more vessels, tanks or the like suitable for carrying out fermentation, as well as associated components and materials required for its operation, as appreciated by persons skilled in the art. The apparatus or system 100 may be configured for continuous processing or batch processing, or partially for continuous processing and partially for batch processing. Accordingly, in the context of an apparatus or system 100 for treating cellulosic material, one or more of the arrows shown in FIG. 1 may represent physical components (e.g., pipes, conduits, containers, or the like) employed for holding the cellulosic material being processed or transporting the cellulosic material from one location or device to another, or may otherwise represent the direction of process flow between locations or devices of the apparatus or system 100.

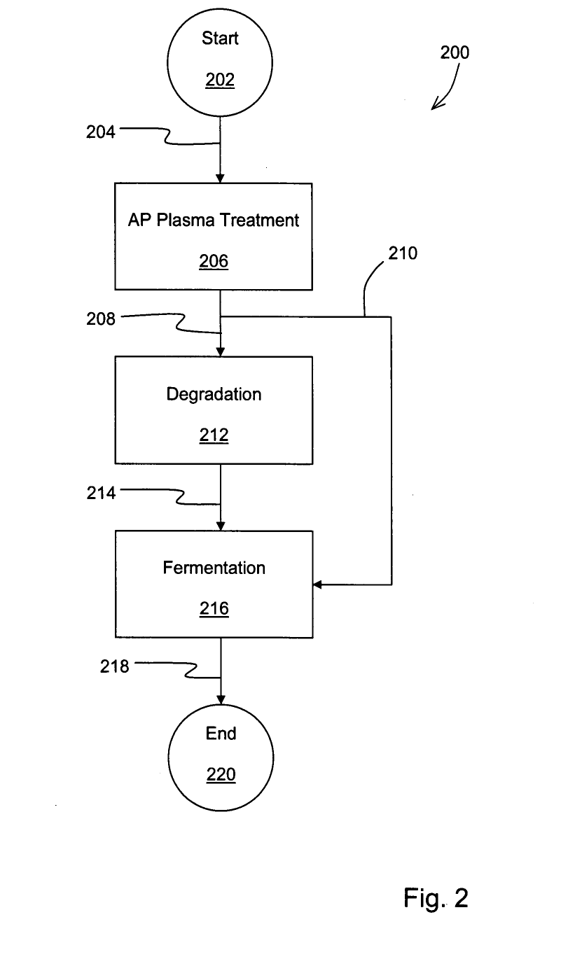

FIG. 2 is a flow diagram 200 illustrating another example of a method for treating cellulosic material. The flow diagram 200 may also represent an apparatus or system capable of performing the illustrated method.

The method begins at the starting point 202. As in the case of the method illustrated in FIG. 1, the starting point 202 may be representative of any suitable preliminary steps taken to prepare the cellulosic material for treatment by AP plasma. The raw or prepared cellulosic material 204 is introduced to an AP plasma apparatus. At block 206, the AP plasma apparatus is operated to generate and maintain a suitable AP plasma that interacts with the cellulosic material 204. The process conditions (pressure, temperature, duration, etc.) of the AP plasma treatment 206 may be the same as or similar to those described above for the method illustrated in FIG. 1.

The treatment of the cellulosic material 204 by AP plasma at block 206 results in an AP plasma-treated cellulosic material 208. As described above, the plasma-treated cellulosic material 208 may include oligosaccharide and/or monosaccharide species released from components of the cellulosic material (cellulose and/or hemicellulose) as a result of the AP plasma treatment 206, as well as residual polysaccharide species and lignin not affected by the AP plasma treatment 206. At this stage, if desired, the released sugars may be recovered and separated from the plasma-treated cellulosic material 208 by any suitable means and then subjected to further processing as necessary to provide commercial-grade sugars. Alternatively, as indicated by the schematic process line 210 in FIG. 2, the released sugars may be recovered for subsequent fermentation.

The portion of the plasma-treated cellulosic material 208 that has not been degraded by the AP plasma treatment step 206 is nevertheless, as a result of the AP plasma treatment step 206, optimally conditioned for subsequent degradation processing. Accordingly, at block 212, the plasma-treated cellulosic material 208 may then be subjected to any suitable cellulosic material degradation or depolymerization process. The degradation process 212 may be any process suitable for yielding desired sugars such that the sugars may then be subsequently processed for commercial consumption or fermented for producing alcohols or other chemicals. Examples of suitable degradation processes 212 include, but are not limited to, acid hydrolysis processes and enzymatic hydrolysis processes. Acid hydrolysis generally entails reacting the plasma-treated cellulosic material 208 with water and employing a suitable acid or acidic compound as a catalyst. Examples of suitable acids and acidic or acid-like compounds include, but are not limited to, mineral acids such as sulfuric acid, sulfurous acid, hydrochloric acid, hydrofluoric acid, phosphoric acid, formic acid and nitric acid, and acidic salts such as aluminum sulfate, ferric sulfate, ferrous sulfate, magnesium sulfate, ferric chloride, aluminum chloride, aluminum nitrate, and ferric nitrate. More generally, a variety of organic and inorganic acids may be employed as appreciated by persons skilled in the art, but particularly toxic or corrosive acids should be avoided. Enzymatic hydrolysis generally entails reacting the plasma-treated cellulosic material 208 with one or more appropriate carbohydrase enzymes such as various known cellulases and hemicellulases. For instance, a cellulase enzyme complex may be employed for the saccharification of the cellulose of the plasma-treated cellulosic material 208 to yield glucose.

The degradation process 212 results in (at least partially) degraded cellulosic material 214. The degraded (or degradation-processed) cellulosic material 214 may include oligosaccharide and/or monosaccharide species released from components of the cellulosic material (cellulose and/or hemicellulose) as well as residual polysaccharide species and lignin not affected by the degradation process 212. Due to the preceding AP plasma treatment 206, the degradation process 212 may result in a much higher yield of monosaccharides than had the degradation process 212 been carried out alone without the AP plasma treatment 206 or had the degradation process 212 been preceded by a conventional pre-treatment process. At this stage, if desired, the released sugars may be recovered and separated from the degraded cellulosic material 214 by any suitable means and then subjected to further processing as necessary to provide commercial-grade sugars. In the case where sugars are the intended end product, the method ends at 220.

In other implementations, as illustrated in FIG. 2, the process may continue by subjecting the sugars obtained from the degraded cellulosic material 214 to any suitable fermentation processing 216 to produce a fermentation product 218. The fermentation product 218 may include alcohols such as ethanol and/or other desired chemicals. As described above in conjunction with the method exemplified in FIG. 1, the fermentation process 216 may entail the use of any microorganisms capable of converting the sugars (e.g., oligosaccharides, monosaccharides, and the like) into the desired alcohols or other compounds. Moreover, it will be understood that the fermentation process 216 may be preceded by any suitable pre-conditioning steps deemed necessary in preparation for fermentation, such as neutralization, removal of any components deemed to act as fermentation inhibitors, and the like. Furthermore, the fermentation product 218 may be subjected to any suitable post-fermentation processes as needed, such as distillation to separate the desired alcohols or other chemicals from the fermentation medium and purify the alcohols or other chemicals for commercially-acceptable uses. The method ends at 220.

As noted above, FIG. 2 may also represent an example of an apparatus or system 200 for treating cellulosic material. Accordingly, block 206 may be considered as depicting a means for subjecting cellulosic material to AP plasma. Examples of such means are referred to above in connection with the apparatus or system 100 illustrated in FIG. 1. Block 212 may be considered as depicting a means for degrading cellulosic material to produce sugars. An example of such means is one or more vessels, tanks or the like suitable for carrying out a degradation process such as, for example, acid or enzymatic hydrolysis, as well as associated components and materials required for its operation, as appreciated by persons skilled in the art. Block 216 may be considered as depicting a means for fermenting plasma-treated cellulosic material. As previously noted, an example of such means is one or more vessels, tanks or the like suitable for carrying out fermentation, as well as associated components and materials required for its operation. The apparatus or system 200 may be configured in whole or part for continuous processing or batch processing, as noted above in connection with the apparatus or system 100 illustrated in FIG. 1.

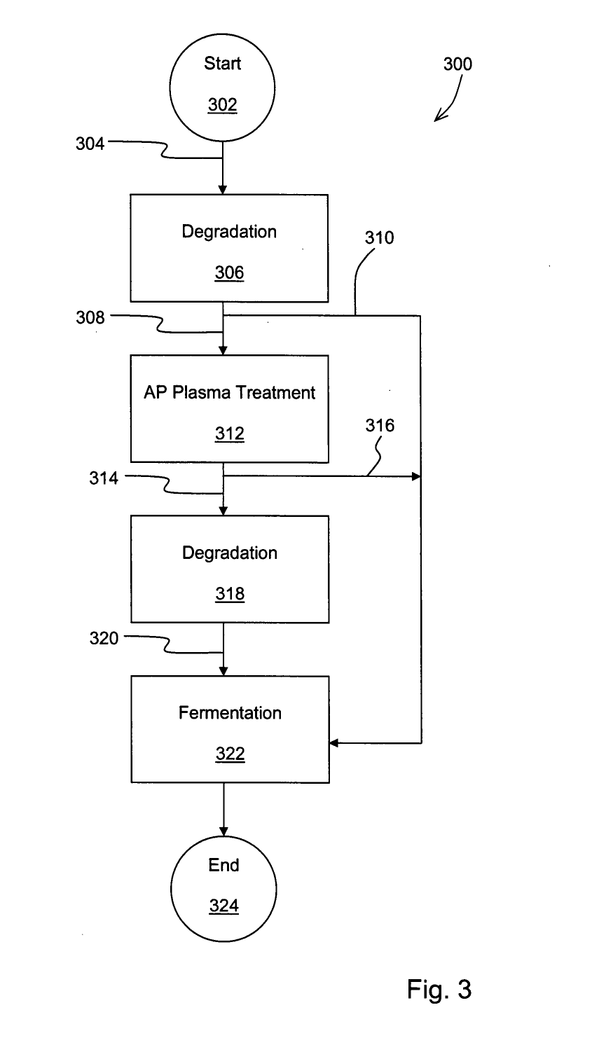

Prior to fermentation, more than one iteration of AP treatment and/or degradation may be performed. For example, FIG. 3 is a flow diagram 300 illustrating another method for treating a cellulosic material. The flow diagram 300 may also represent an apparatus or system capable of performing the illustrated method. This example entails a two-stage degradation process that is enhanced by one or more AP plasma treatment steps.

The method begins at the starting point 302. Raw or prepared cellulosic material 304 is subjected to a first-stage degradation process at block 306, in which at least some of the components of the cellulosic material 304 are degraded or depolymerized without the aid of AP plasma treatment. For example, the first-stage degradation process 306 may entail dilute acid hydrolysis. The first-stage degradation process 306 may serve as a relatively mild process that acts on certain polysaccharide components of the cellulosic material 304 that, due to their initial structure (e.g., degree of crystallinity or amorphousness) or accessibility (e.g., exposure, freedom from lignin binding, etc.), are readily degradable without the aid of a pre-treatment step. Alternatively, or additionally, the first-stage degradation process 306 may serve as a pre-treatment process in and of itself, for example to break down the hemicellulose for removal, and/or more generally to at least partially disrupt the cellulose-hemicellulose-lignin complex, in preparation for hydrolyzing or otherwise degrading the cellulose (and particularly the crystalline phase) in a subsequent degradation step.

The first-stage degradation process 306 produces (at least partially) degraded cellulosic material 308, which may be a mixture of sugar solution and residual cellulosic material such as unreacted cellulose and lignin. At this stage, if desired, any suitable separation process may be performed to separate the sugar solution from the residual cellulosic material. The sugars obtained at this stage may be processed for commercial use or, as indicated by line 310 in FIG. 3, recovered for subsequent fermentation. At block 312, the unreacted cellulosic material is subjected to AP plasma treatment as described elsewhere in this disclosure. The process conditions (pressure, temperature, duration, etc.) of the AP plasma treatment 312 may be the same as or similar to conditions described above.

The treatment of the cellulosic material by AP plasma at block 312 results in an AP plasma-treated cellulosic material 314. As described above, the plasma-treated cellulosic material 314 may include oligosaccharide and/or monosaccharide species released from components of the cellulosic material (cellulose and/or hemicellulose) as a result of the AP plasma treatment 312, as well as residual polysaccharide species and lignin not affected by the AP plasma treatment 312. At this stage, if desired, the released sugars may be recovered and separated from the plasma-treated cellulosic material 314 by any suitable means and then subjected to further processing as necessary to provide commercial-grade sugars. Alternatively, as indicated by the schematic process line 316 in FIG. 3, the released sugars may be recovered for subsequent fermentation.

The portion of the plasma-treated cellulosic material 314 that has not been degraded by the AP plasma treatment step 312 is, as a result of the AP treatment step 312, optimally conditioned for subsequent degradation processing. Accordingly, at block 318, the plasma-treated cellulosic material 314 may then be subjected to a second-stage cellulose degradation or depolymerization process. To the extent that the cellulosic material undergoing the second-stage degradation 318 was not degraded into sugars in the first-stage degradation process 306 or AP plasma treatment process 312 and hence is more difficult to degrade, the second-stage degradation process 318 may be a more rigorous process in comparison to the first-stage degradation process 306. For example, the second-stage degradation process 318 may entail an acid hydrolysis process in which a higher concentration of acid is employed as compared with the first-stage acid hydrolysis. For example, the first-stage hydrolysis may be carried out in a 0.5M (4.9% w/w) H₂SO₄ (sulfuric acid) solution, and the second-stage hydrolysis may be carried out in a 1.0M (9.8% w/w) H₂SO₄ solution. Alternatively, depending on the nature of the cellulosic material being processed, the effectiveness of the AP treatment process 312 may be such that the second-stage degradation process 318 need not be more rigorous, or may even be less rigorous, than the first-stage degradation process 306. As another example, the second-stage degradation process 318 may be an enzymatic process that employs enzymes (e.g., cellulases) specifically selected to hydrolyze the more difficultly hydrolyzable components of the cellulosic material such as crystalline cellulose.

The processing of the plasma-treated cellulosic material 314 by the second-stage degradation process 318 yields further degradation-processed cellulosic material 320 that includes sugars. At this stage, the sugars may be subjected to any post-degradation processes such as purification and refinement as necessary to provide commercial-grade sugar. Alternatively, at block 322, the process may continue by subjecting the sugars to any suitable fermentation process to produce alcohols or other desired chemicals. As noted previously, any sugars produced from the first-stage degradation process 306 and the AP plasma treatment process 312 may likewise be fermented. In some implementations, the sugars produced from the first-stage degradation process 306 and/or the AP plasma treatment process 312 may be combined with the sugars produced from the second-stage degradation process 318, and all sugars co-fermented simultaneously. The process illustrated in FIG. 3 ends at 324.

As noted above, FIG. 3 may also represent an example of an apparatus or system 300 for treating cellulosic material. Accordingly, block 306 may be considered as depicting a means for degrading cellulosic material to produce sugars. An example of such means is one or more vessels, tanks or the like suitable for carrying out a degradation process such as, for example, acid or enzymatic hydrolysis, as well as associated components and materials required for its operation. Block 312 may be considered as depicting a means for subjecting cellulosic material to AP plasma. Examples of such means are referred to above in connection with the apparatus or system 100 illustrated in FIG. 1. Block 318 may be considered as depicting a separate degrading means, or may be the same means as means 306. Block 322 may be considered as depicting a means for fermenting plasma-treated cellulosic material. As previously noted, an example of such means is one or more vessels, tanks or the like suitable for carrying out fermentation, as well as associated components and materials required for its operation. The apparatus or system 300 may be configured in whole or part for continuous processing or batch processing, as noted above in connection with the apparatus or system 100 illustrated in FIG. 1.

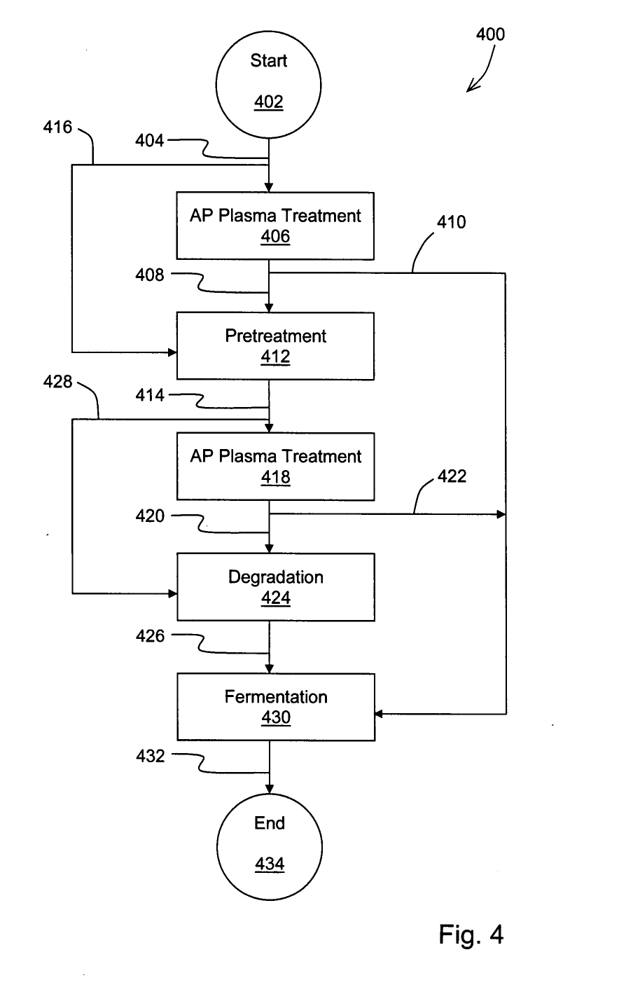

Depending on the composition of the cellulosic material to be processed, one or more AP plasma treatment steps may be combined with one or more degradation processes, as well as with “pre-treatment” processes traditionally associated with conventional degradation processes such as acid hydrolysis and enzymatic hydrolysis. The pre-treatment process may be any chemical, biological, biochemical, physical, or physio-chemical process or processes now known or later developed that is effective in enhancing conventional degradation processes. Examples of pre-treatment processes include, but are not limited to, comminution, uncatalyzed steam explosion, hydrothermolysis, the addition of acids, bases, solvents, or ammonia, ammonia fiber/freeze explosion (AFEX), ammonia recycled percolation (ARP), etc. An example of a process that combines pre-treatment with AP plasma treatment is illustrated in FIG. 4, which is a flow diagram 400 illustrating another method for treating a cellulosic material. The flow diagram 400 may also represent an apparatus or system capable of performing the illustrated method.

The method begins at the starting point 402. Raw or prepared cellulosic material 404 is introduced to a suitable AP plasma apparatus and subjected to a first-stage AP plasma treatment at block 406, which yields plasma-treated cellulosic material 408 as previously described. As also previously described, the plasma-treated cellulosic material 408 may include sugars as a result of the AP plasma treatment 406, as well as residual polysaccharide species and lignin not affected by the AP plasma treatment 406. At this stage, if desired, the released sugars may be recovered and separated from the plasma-treated cellulosic material 408 by any suitable means and then subjected to further processing as necessary to provide commercial-grade sugars. Alternatively, as indicated by the process schematic line 410 in FIG. 4, the sugars resulting from the first-stage AP plasma treatment 406 may be recovered for subsequent fermentation. At block 412, the plasma-treated cellulosic material 408 is subjected to any suitable pre-treatment process to enhance a subsequent degradation technique or techniques. The pre-treatment process 412 yields pre-treated cellulosic material 414.

In other implementations, as indicated by the process schematic line 416, raw or prepared cellulosic material 404 is subjected directly to the pre-treatment process 412 without an intervening AP plasma treatment 406.

The pre-treated cellulosic material 414 is then subjected to a second-stage AP plasma treatment at block 418, which yields further plasma-treated cellulosic material 420. The second-stage AP plasma treatment 418 may serve to enhance the role of the pre-treatment step 412 (and the first-stage AP plasma treatment 406, if employed) in optimizing the cellulosic material for a subsequent degradation process or processes. On the other hand, the pre-treatment step 412 may be considered as enhancing the role of the first-stage AP plasma treatment 406 and/or the second-stage AP plasma treatment 418 in optimizing the cellulosic material for subsequent degradation. Moreover, as in the case of the first-stage AP plasma treatment 406, the second-stage AP plasma treatment 418 may yield sugars as a result of second-stage AP plasma treatment 418. At this stage, if desired, these sugars may be recovered and separated from the plasma-treated cellulosic material 420 by any suitable means and then subjected to further processing as necessary to provide commercial-grade sugars. Alternatively, as indicated by the process schematic line 422 in FIG. 4, the sugars resulting from the second-stage AP plasma treatment 418 may be recovered for subsequent fermentation.

After the second-stage AP plasma treatment 418, at block 424, the plasma-treated cellulosic material 420 is subjected to any suitable degradation process such as, for example, acid hydrolysis or enzymatic hydrolysis to break down remaining polysaccharide components of the plasma-treated cellulosic material 420 into sugars. The degradation process 424 yields a mixture 426 of sugar solution and residual cellulosic material such as lignin.

In other implementations, as indicated by the process schematic line 428, the pre-treated cellulosic material 414 is subjected directly to the degradation process 424 without an intervening AP plasma treatment 418.

At this stage, any suitable separation process may be performed to separate the sugar solution from the residual cellulosic material. The sugars obtained at this stage may be processed for commercial use. Alternatively, as indicated at block 430, the resulting sugars may be subjected any suitable fermentation process to produce a fermentation product 432 that includes alcohols or other desired chemicals. As noted previously, any sugars produced from the first-stage AP plasma treatment 406 and/or the second-stage AP plasma treatment 418 may likewise be fermented, together with or separately from the sugars derived from the degradation process 424. The process illustrated in FIG. 4 ends at 434.

As noted above, FIG. 4 may also represent an example of an apparatus or system 400 for treating cellulosic material. Accordingly, block 406 may be considered as depicting a means for subjecting cellulosic material to AP plasma. Examples of such means are referred to above in connection with the apparatus or system 100 illustrated in FIG. 1. Block 412 may be considered as depicting a means for pre-treating cellulosic material in preparation for a degradation process. Examples of pre-treatment processes are noted above, and the devices and systems for implementing such pre-treatment processes are known to persons skilled in the art. Block 418 may be considered as depicting a separate AP plasma treatment means, or may be the same means as means 406. Block 424 may be considered as depicting a means for degrading cellulosic material to produce sugars. An example of such means is one or more vessels, tanks or the like suitable for carrying out a degradation process such as, for example, acid or enzymatic hydrolysis, as well as associated components and materials required for its operation. Block 430 may be considered as depicting a means for fermenting plasma-treated cellulosic material. As previously noted, an example of such means is one or more vessels, tanks or the like suitable for carrying out fermentation, as well as associated components and materials required for its operation. The apparatus or system 400 may be configured in whole or part for continuous processing or batch processing, as noted above in connection with the apparatus or system 100 illustrated in FIG. 1.

Any of the methods, apparatus and systems described by example above and illustrated by example in FIGS. 1-4, in implementations involving fermentation, may be adapted for separate hydrolysis and fermentation (SHF), simultaneous saccharification and fermentation (SSF), and/or simultaneous saccharification co-fermentation (SSCF).

FIGS. 5-9 illustrate examples of AP plasma apparatus or systems that may be employed in the AP plasma treatment processes performed in methods according to the invention, including the methods described by above example and illustrated by example in FIGS. 1-4. It will be understood that implementations of AP plasma apparatus or systems other than those illustrated in FIGS. 5-9 may also be suitable in the practice of the invention.

Referring now to FIG. 5, an AP plasma apparatus or system 500 is illustrated. The AP plasma apparatus 500 may be provided as a dielectric barrier discharge (DBD) apparatus. The AP plasma apparatus 500 may include a housing or enclosure 504 in which AP plasma treatment of cellulosic material 508 is performed. It will be understood, however, that because the plasma operates at atmospheric pressure, an enclosure 504 is not required, at least not one of the type required in evacuated or pressurized systems. The AP plasma apparatus 500 has a parallel-plate configuration, and thus includes a first electrode 512 and a second electrode 516 spaced at a distance from the first electrode 512. The first and second electrodes 512 and 516 may be positioned or mounted within the enclosure 504 by any suitable means. Although in the present example the electrodes 512 and 516 are illustrated as having a planar geometry (i.e., plates), any other suitable geometry may be employed for the electrodes 512 and 516. For example, a cylindrical or rectangular symmetry may be employed. The electrodes 512 and 516 may be composed of any suitable electrically conductive material such as, for example, aluminum (Al). One or both of the electrodes 512 and 516 may be coated, covered, or otherwise isolated by a dielectric element (e.g., a layer, film, or the like). In the present example, the first electrode 512 is covered by a first dielectric element 520 and the second electrode 516 is covered by a second dielectric element 524. The dielectric elements 520 and 524 may be composed of any suitable electrically insulating material such as, for example, a silica glass. A high-voltage power supply 528 electrically communicates with the first and second electrodes 512 and 516 via suitable conductive elements 532 and 536, respectively. The electrodes 512 and 516 are separated by some distance, typically less than 25.4 mm. The gap is filled with suitable gas at a pressure at or near 760 Torr but may range from 100 to 1000 Torr as noted previously. Due to the presence of one or more dielectric elements 520 and 524 between the electrodes 512 and 516, the high-voltage power supply 528 is an AC (alternating current) source. Although in this configuration two dielectric elements 520 and 524 are employed, it is not required that both electrodes 512 and 516 be covered in this manner. It has been shown that materials with a high resistivity can also act as a barrier for plasma formation. Additionally, in other implementations the power source can be operated in other modes such as a DC, pulsed DC, or RF voltage (up to and including the Ghz range in frequency), etc. as previously noted. In the present configuration, the high-voltage power supply 528 may operate at an amplitude ranging from about 500 V to about 50,000 V and a frequency ranging from about 0.050 kHz to about 150 kHz.

In operation, a mass of cellulosic material 508 is introduced into the AP plasma apparatus 500 by any means and positioned between the electrodes 512 and 516 and dielectric barrier(s) 520 and 524. The cellulosic material 508 introduced into the AP plasma apparatus 500 may be dry, moist or combined with a liquid medium (e.g., water). In the example illustrated in FIG. 5, the cellulosic material 508 is provided on or in a suitable holder 540. The holder 540 may, for example, be any container, planar surface or platter utilized for conventional applications (e.g., semiconductor or micro-electro-mechanical fabrication) of plasma-generating apparatus. One non-limiting example of a holder 540 is a fluoroware container. One or more working gases suitable for generating a plasma such as, for example, argon and/or helium, are then introduced into the interior of the enclosure 504. Alternatively, ambient air may be employed as the plasma medium, in which case a specific gas introduction step is not required. The high-voltage power supply 528 is then operated to strike a bulk plasma 544 between the electrodes 512 and 516 and dielectric barrier(s) 520 and 524. In the present context, the term “bulk” indicates a wide-beam or non-directional plasma 544 that maximizes contact with the cellulosic material 508. That is, a focused narrow plasma beam is not required in this implementation but may be employed in other implementations utilizing an AP plasma. As appreciated by persons skilled in the art, the high-voltage power supply 528 may be operated according to a voltage profile suitable for the design of the AP plasma apparatus 500 and the working gas employed under conditions suitable for generation of an AP plasma. For instance, the amplitude of the voltage applied to the electrodes 512 and 516 is typically relatively high to strike the plasma 544, and subsequently is lowered to maintain the plasma 544. It will also be noted that the plasma 544 may be generated prior to introduction of the cellulosic material 508 into the AP plasma apparatus 500. Other operating conditions of the AP plasma apparatus 500, such as temperature and time duration of the treatment, may fall within the ranges noted above in conjunction with the method illustrated in FIG. 1. The exposure of the cellulosic material 508 to the energetic AP plasma 544 results in a plasma-treated cellulosic material, the effects of which are described above in conjunction with the examples of methods illustrated in FIGS. 1-4.

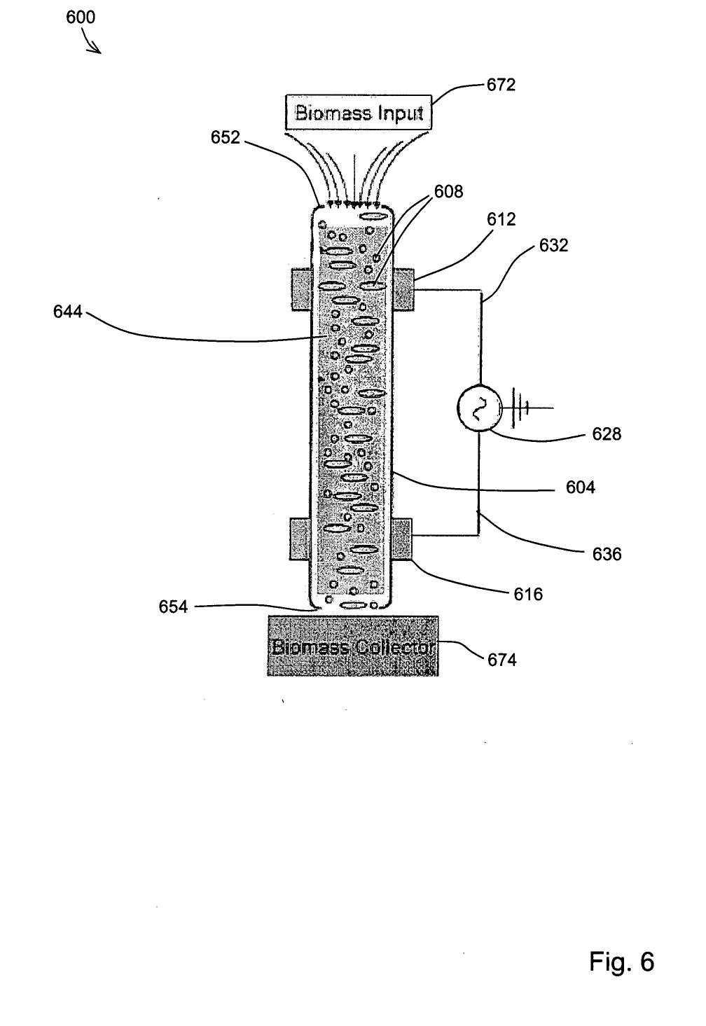

FIG. 6 illustrates another AP plasma apparatus or system 600, which may be provided as a DBD apparatus. The AP plasma apparatus 600 includes a housing, container or enclosure 604 in which AP plasma treatment of cellulosic material 608 is performed. Moreover, the enclosure 604 may be composed of a glass or other suitable electrically insulating material such as Pyrex® glass or other silicate or borosilicate glass, quartz or alumina, thereby serving as a dielectric barrier as well as a plasma reaction chamber. It will be understood, however, that implementations of the invention are not limited to operation in a reaction chamber, as the plasma may be operated under atmospheric pressure as previously noted. The enclosure 604 may be generally cylindrical or have some other hollow geometry. As illustrated in FIG. 6, the enclosure 604 may be vertically oriented as a drop tube and thus elongated along the vertical dimension. Accordingly, the AP apparatus 600 may be referred to herein as having a drop-tube configuration. In other implementations, the enclosure 604 may be horizontally oriented or oriented at an angle relative to a vertical or horizontal plane. The AP plasma apparatus 600 also includes a first electrode 612 and a second electrode 616 spaced at a distance from the first electrode 612. The electrodes 612 and 616 may be composed of any suitable electrically conductive material such as, for example, aluminum (Al). The electrodes 612 and 616 are coaxially disposed around the outside surface of the enclosure 604 and fixed in position by any means. The electrodes 612 and 616 may be entirely or partially formed as annular or ring-shaped members. A high-voltage AC power supply 628 electrically communicates with the first and second electrodes 612 and 616 via suitable conductive elements 632 and 636, respectively. The high-voltage power supply 628 may operate at an amplitude ranging from about 500 to about 50,000 V and a frequency ranging from about 0.05 to about 150 kHz.

The enclosure 604 has an opening at one end or, in the flow-through implementation illustrated in FIG. 6, has an inlet opening 652 at one end and an outlet opening 654 at the opposite end. In the illustrated vertically-oriented implementation, the AP plasma apparatus 600 may additionally include a container or holder 672 such as a hopper or other suitable design that is positioned above the inlet opening 652 of the enclosure 604 for initially containing untreated cellulosic material 608 and thereafter dropping the cellulosic material 608 by gravity feed into the interior of the enclosure 604 via the inlet opening 652. The AP plasma apparatus 600 may also include another holder or container 674 positioned below the outlet opening 654 of the enclosure 604 for collecting AP plasma-treated cellulosic material 608. Alternatively, opening 654 may serve as the inlet opening and opening 652 may serve as the outlet opening, or one of openings 652 or 654 may serve as both the inlet opening and the outlet opening.

In operation, one or more working gases suitable for generating a bulk plasma 644 such as, for example, argon and/or helium, are introduced into the interior of the enclosure 604. Alternatively, ambient air may be employed as the plasma medium. The high-voltage power supply 628 is operated to strike the bulk plasma 644 within the chamber defined by the enclosure 604. The operating conditions of the AP plasma apparatus 600, such as the voltage profile of the high-voltage power supply 628, the temperature within the enclosure 604, and the time duration of the treatment, may be as noted above in conjunction with the method illustrated in FIG. 1 and the apparatus 500 illustrated in FIG. 5. A mass of cellulosic material 608 is introduced into the enclosure 604 such as by unloading the holder 672 and allowing the cellulosic material 608 to flow through the inlet opening 652 by gravity feed. The cellulosic material 608 introduced into the AP plasma apparatus 600 may be dry or moist. In this implementation, it is desirable in many cases that the cellulosic material 608 be comminuted sufficiently to maximize the duration of travel through the enclosure 604 during operation of the AP plasma 644. The cellulosic material 608 is thus treated by the energetic AP plasma 644 as it flows down through the plasma 644 along the elongated dimension of the enclosure 604. At the bottom of the enclosure 604, the resulting plasma-treated cellulosic material 608 flows through the outlet opening 654 and into the holder 674 for collection.

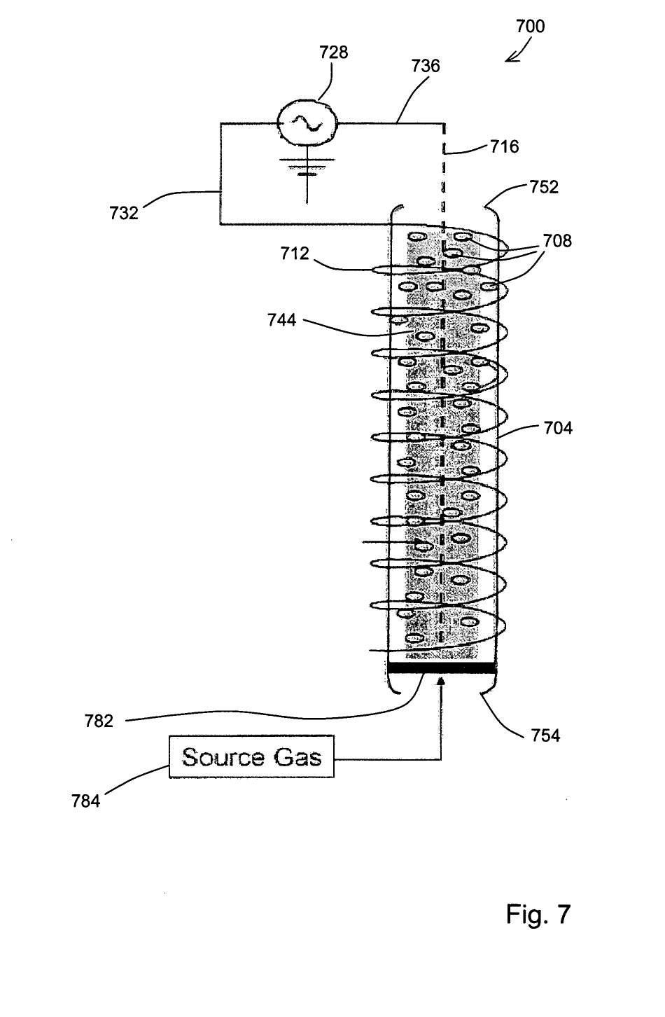

FIG. 7 illustrates another AP plasma apparatus or system 700, which may be provided as a DBD apparatus. The AP plasma apparatus 700 includes a housing, container or enclosure 704 in which AP plasma treatment of cellulosic material 708 is performed. Similar to the apparatus 600 shown in FIG. 6, the enclosure 704 may be composed of a glass or other suitable electrically insulating material and hence function as a dielectric barrier as well as a plasma reaction chamber. The enclosure 704 may be generally cylindrical or have some other hollow geometry, and may be vertically oriented and elongated. The AP plasma apparatus 700 also includes a first electrode 712 and a second electrode 716 spaced at a distance from the first electrode 712. The electrodes 712 and 716 may be composed of any suitable electrically conductive material such as, for example, aluminum (Al), and may be elongated members. The first electrode 712 is helically wound as a coil around the outside surface of the enclosure 704 or otherwise coaxially disposed about the enclosure 704. The second electrode 716 extends as a rod or wire into the enclosure 704 generally along the central longitudinal axis of the interior of the enclosure 704. A high-voltage AC power supply 728 electrically communicates with the first and second electrodes 712 and 716 via suitable conductive elements 732 and 736, respectively. The high-voltage power supply 728 may operate at an amplitude ranging from about 500 to about 50,000 V and a frequency ranging from about 0.05 to about 150 kHz.

The enclosure 704 has an opening at one end or, in the flow-through implementation illustrated in FIG. 7, has an inlet opening 752 at one end and an outlet opening 754 at the opposite end. The AP plasma apparatus 700 may additionally include a container or holder (not shown, but see FIG. 6) such as a hopper or other suitable design that is positioned above the inlet opening 752 of the enclosure 704 for initially containing untreated cellulosic material 708 and thereafter dropping the cellulosic material 708 by gravity feed into the interior of the enclosure 704 via the inlet opening 752. The AP plasma apparatus 700 may also include another holder or container (not shown, but see FIG. 6) positioned below the outlet opening 754 of the enclosure 704 for collecting AP plasma-treated cellulosic material 708. Alternatively, as described below, the opening 754 may serve as the inlet opening and the opening 752 may serve as the outlet opening, or one of the openings 752 or 754 may serve as both the inlet opening and the outlet opening.

Additionally, a gas distributor 782 is positioned by any means in the bottom region of the enclosure 704 near the outlet opening 754. The gas distributor 782 may have any configuration suitable for flowing a gas up through the interior of the enclosure 704. For example, the gas distributor 782 may include a manifold and a plurality or orifices or jets (not shown) for this purpose. The gas distributor 782 may be annular or toroidal in shape, or have a plurality of passages extending from the upper side of the gas distributor 782 to the lower side, to facilitate a flow-through implementation of the AP plasma apparatus 700, i.e., to allow plasma-treated cellulosic material 708 to flow through a center opening or plurality of passages provided by the gas distributor 782. The gas distributor 782 communicates with a suitable gas source 784. By this configuration, the AP plasma apparatus 700 may be considered as operating as a fluidized-bed reactor. For this purpose, any suitable gas may be supplied to the gas distributor 782. Examples of gases include, but are not limited to, air, oxygen, hydrogen, helium, water-saturated helium, neon, argon, hydrogen, nitrogen, xenon, carbon dioxide, SF₆, CF₄, NH₃ and combinations of two or more of the foregoing.

In operation, one or more working gases suitable for generating a bulk plasma 744 such as, for example, argon and/or helium, are introduced into the interior of the enclosure 704. The gas distributor 782 may be employed for this purpose, or additional, dedicated working gas supply and delivery means (not shown) may be provided. Alternatively, ambient air may be employed as the plasma medium. The high-voltage power supply 728 is operated to strike the bulk plasma 744 within the chamber defined by the enclosure 704. The operating conditions of the AP plasma apparatus 700, such as the voltage profile of the high-voltage power supply 728, the temperature within the enclosure 704, and the time duration of the treatment, may be as noted above in conjunction with the method illustrated in FIG. 1 and the apparatus 500 illustrated in FIG. 5. A flow of gas is established through the gas distributor 782 into the interior of the enclosure 704, with the gas being directed generally in the upward vertical direction. A mass of cellulosic material 708 is introduced into the enclosure 704 such as by unloading a holder (not shown) and allowing the cellulosic material 708 to flow through the inlet opening 752 by gravity feed. Alternatively, the cellulosic material 708 may be introduced at some flow rate into the enclosure 704 through opening 754 (if provided) or some other opening near the bottom of the enclosure 704, and conducted upward through the enclosure 704 with the assistance of the gas flow. The cellulosic material 708 introduced into the AP plasma apparatus 700 may be dry or moist. In this implementation, it is desirable in many cases that the cellulosic material 708 be comminuted sufficiently to maximize the duration of travel through the enclosure 704 during operation of the AP plasma 744. In such a case, however, the cellulosic material 708 need not be comminuted to the same degree as in the case of the apparatus 600 shown in FIG. 6, because the gas distributor 782 establishes a fluidized-bed condition in the enclosure 704 by which the cellulosic material 708 is suspended for a suitable working time due to a balancing of forces from gravity and the gas flow. Moreover, the fluidized-bed condition may cause the components of the cellulosic material 708 to flow along turbulent or tumbling pathways, thereby enabling more effective interaction between the cellulosic material 708 and the plasma 744. The cellulosic material 708 is thus treated by the energetic AP plasma 744 as it is effectively suspended in and flows through the plasma 744. At the bottom of the enclosure 704, the resulting plasma-treated cellulosic material 708 flows through or around the gas distributor 782, through the outlet opening 754, and optionally into a holder (not shown) for collection. In other implementations, the flow of gas through the gas distributor 782 may be increased so as to over-pressurize the enclosure 704 and thereby conduct the plasma-treated cellulosic material 708 out through the inlet opening 752 of the enclosure 704 and into a suitable collector.

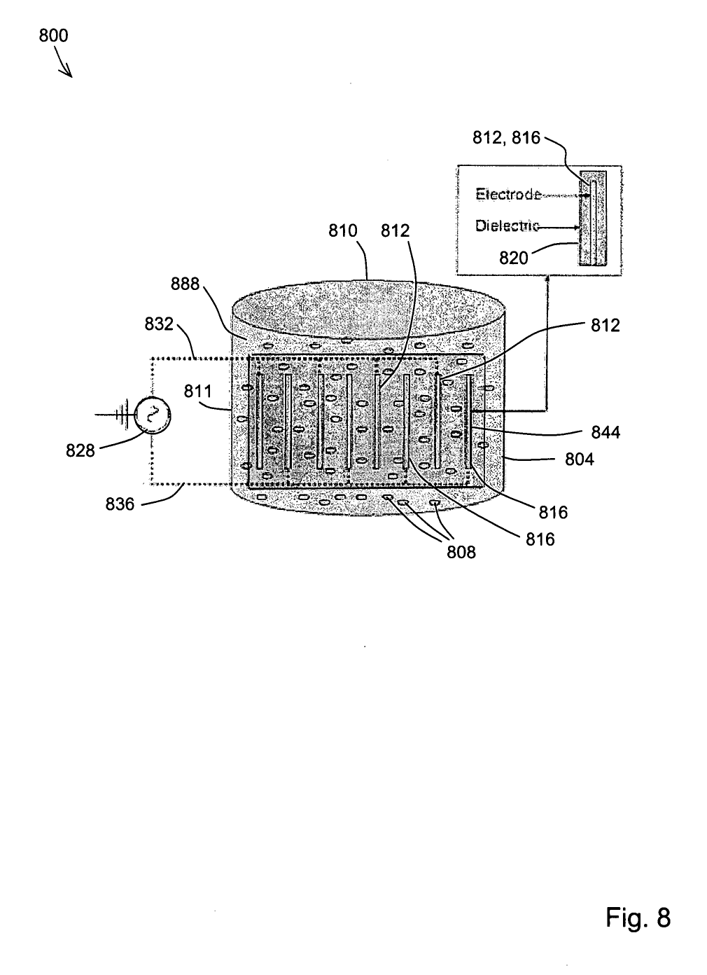

FIG. 8 illustrates another AP plasma apparatus or system 800. The AP plasma apparatus 800 includes a housing, container or enclosure 804 in which AP plasma treatment of cellulosic material 808 is performed. The enclosure 804 may be provided as a container with an open or closed top 810 and contain a volume of liquid 888 thereby serving as a liquid bath. Accordingly, the AP plasma apparatus 800 may be referred to as having a liquid-bath configuration. Examples of liquids 888 include, but are not limited to, water, acids, bases, and solvents. The enclosure 804 may be generally cylindrical or have some other hollow geometry suitable for holding the volume of liquid 888. The AP plasma apparatus 800 also includes one or more electrodes 812 and 816 spaced at distances from each other. The distance intervals between adjacent electrodes 812 and 816 may be equal or non-equal. The electrodes 812 and 816 may be composed of any suitable electrically conductive material such as, for example, aluminum (Al). The electrodes 812 and 816 may be elongated members such as wires or rods, may be plates, or may have any other suitable shapes. The electrodes 812 and 816 may be fixed in position within the interior of the enclosure 804 by any means, and thus in practice are immersed in the liquid bath. The electrodes 812 and 816 may be vertically oriented as in the illustrated example or positioned in any other orientation. As illustrated in the inset portion of FIG. 8, one or more of the electrodes 812 and 816 may be coated or otherwise covered with a dielectric layer 820 composed of any suitable electrically insulating material such as, for example, Pyrex® glass or other silicate or borosilicate glass, quartz, or alumina. A high-voltage AC power supply 828 electrically communicates with the first set of electrodes 812 and second set of electrodes 816 via suitable conductive elements 832 and 836, respectively. The conductive elements 832 and 836 may be passed through a sidewall 811 of the enclosure 804 such as by employing sealed feed-through members (not shown). Alternatively, in the case where the top 810 of the enclosure 804 is open, the conductive elements 832 and 836 may be routed into the interior of the enclosure 804 via the open top 810. The high-voltage power supply 828 may operate at an amplitude ranging from about 3000 to about 50,000 V and a frequency ranging from about 1 to about 150 kHz.

In operation, the enclosure 804 is partially or completely filled with a volume of liquid 888, which serves as the plasma medium. A mass of cellulosic material 808 is introduced into the liquid bath by any means and permitted to become distributed through the volume of liquid 888, at least in the region of the electrodes 812 and 816 where the plasma is generated The high-voltage power supply 828 is operated to strike a bulk plasma 844 within the enclosure 804. The operating conditions of the AP plasma apparatus 800, such as the voltage profile of the high-voltage power supply 828, the temperature within the enclosure 804, and the time duration of the treatment, may be as noted above in conjunction with the method illustrated in FIG. 1 and the apparatus 500 illustrated in FIG. 5. In some implementations, the plasma 844 may be generated prior to introducing the cellulosic material 808, but it may desirable to first introduce the cellulosic material 808 to allow the cellulosic material 808 to become distributed prior to treatment by the plasma 844. The cellulosic material 808 is thus treated by the energetic AP plasma 844 for a desired period of time (e.g., 5 minutes or thereabouts). The resulting plasma-treated cellulosic material 808 is thereafter removed from AP plasma apparatus 800, and may be subjected to further processing in accordance with any of the methods of the invention.

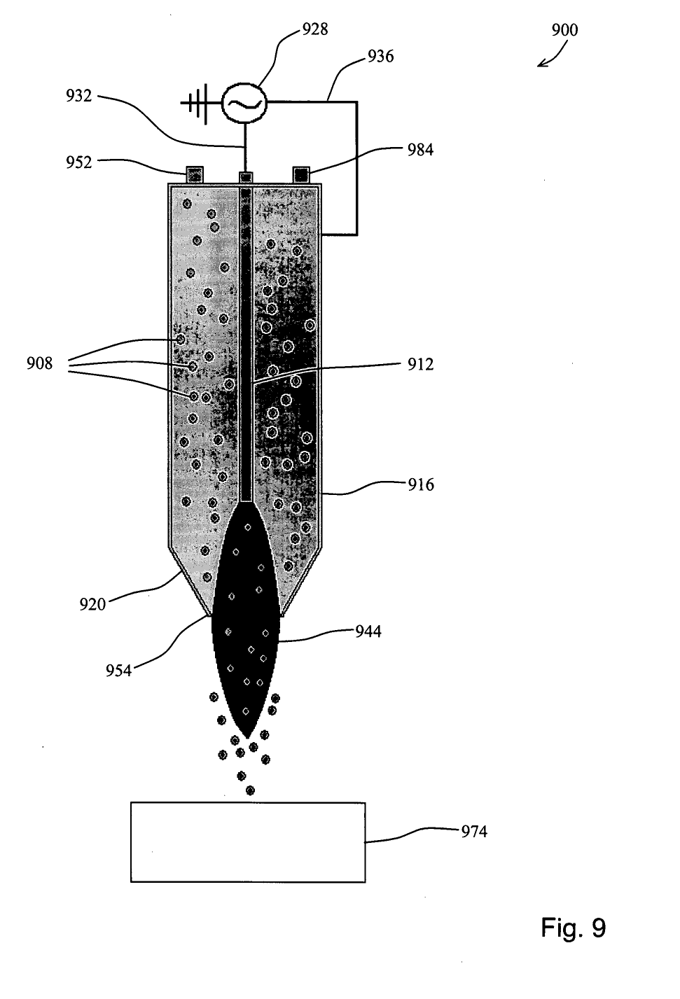

FIG. 9 illustrates another AP plasma apparatus or system 900. The AP plasma apparatus 900 includes a first electrode 912 and an encasement or enclosure 916. At least a portion of the enclosure 916 serves as a second electrode. The first electrode 912 and the enclosure 916 (or at least the electrode portion of the enclosure 916) may be composed of any suitable electrically conductive material such as, for example, aluminum. A high-voltage AC power supply 928 electrically communicates with the first electrode 912 and the enclosure 916 via suitable conductive elements 932 and 936, respectively. The enclosure 916 provides an internal volume for containing cellulosic material 908, which may be fed into the enclosure 916 via an inlet opening 952 typically located at or near the top of the enclosure 916. The first electrode 912 extends through the top of the enclosure 916 and into the interior volume, typically along the central, longitudinal axis of the enclosure 916. The first electrode 912 terminates at some point within the enclosure 916 and is radially spaced from an annular or hollow, conductive portion of the enclosure 916. The cross-section of the enclosure 916 may be generally circular, elliptical, or polygonal. The lower region of the enclosure 916 may be tapered so as to form a nozzle section 920 that terminates at an outlet opening 954.

In operation, the cellulosic material 908 is fed into the enclosure 916 through the inlet opening 952. A source gas for generating AP plasma is fed through a source gas inlet opening 984 of the enclosure 916. The cellulosic material 908 and source gas flow generally from the top of the enclosure 916 toward the nozzle section 920. The high-voltage power supply 928 is operated to ignite the source gas, thereby creating an ignited plasma 944 in the nozzle section 920 of the enclosure 916 in the form of a plasma jet, which is directed out from the outlet opening 954 of the nozzle section 920. The cellulosic material 908 is treated by the plasma 944 as it flows with the plasma 944 out from the outlet opening 954. The treated cellulosic material 908 is collected at a suitable collector 974, which may be positioned at some distance from the outlet opening 954.

In at least some implementations and for at least some types of cellulosic materials, the apparatus 600, 700, 800 and 900 respectively illustrated in FIGS. 6, 7, 8 and 9 may be more effective at treating cellulosic materials as compared with the apparatus 500 illustrated in FIG. 5, due to the three-dimensional exposure of the cellulosic material to the plasma.

In addition to the implementations described above and illustrated in FIGS. 6-9, another type of plasma-generating apparatus that may be employed is one configured for generating a microplasma. The design and operation of microplasma-generating apparatus, outside of the context of treating cellulosic materials, are known to persons skilled in the art. One of the above-described apparatus 500, 600, 700, 800 and 900 may be configured to produce a microplasma.

### EXAMPLE 1

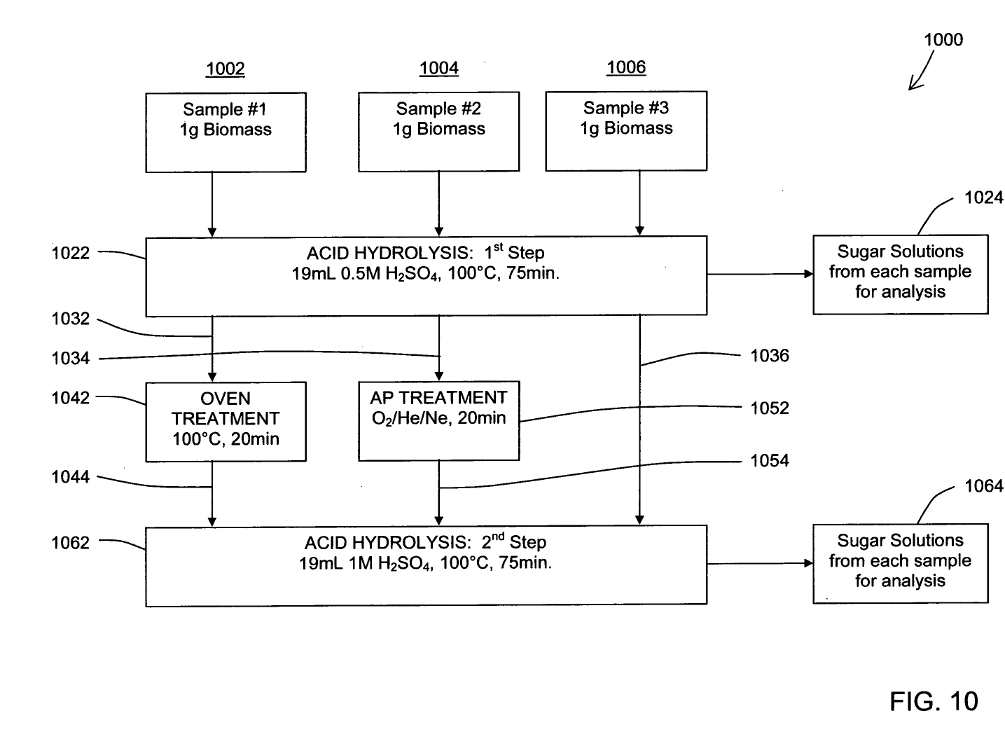

FIG. 10 is a schematic diagram 1000 illustrating an experimental implementation carried out to determine the impact of treatment by AP plasma on the process of acid hydrolysis of cellulosic material. In this experiment, the cellulosic material studied was biomass, specifically maple sawdust. In FIG. 10, the samples are designated Sample #1, Sample #2, and Sample #3. Each of Sample #1, Sample #2, and Sample #3 consisted of 1 gram (g) of dry maple sawdust. The diagram 1000 illustrates three experimental pathways 1002, 1004 and 1006 to which the three separate but identical samples of maple sawdust were respectively subjected. In the experimental pathways 1002, 1004 and 1006, Sample #1, Sample #2, and Sample #3 were respectively subjected to a conventional, relatively dilute acid hydrolysis process, at block 1022. Each hydrolysis process 1022 entailed placing Sample #1, Sample #2, or Sample #3 in a respective glass container containing a 19 mL mixture of 0.5-M H₂SO₄ (sulfuric acid) and water. An identical container was employed for each of Sample #1, Sample #2, and Sample #3. For each hydrolysis process 1022, the mixture, including the acid, water, and Sample #1, Sample #2, or Sample #3, was heated at 100° C. for 75 minutes. Thereafter, at block 1024, the respective hydrolyzate sugar solutions were removed from each of Sample #1, Sample #2, and Sample #3 for analysis. As expected, the data (e.g., glucose and xylose concentrations) from this analysis was essentially identical for each of Sample #1, Sample #2, and Sample #3. The portions of Sample #1, Sample #2, and Sample #3 not affected by the initial hydrolysis process 1022, designated sample portions 1032, 1034 and 1036, respectively, were subjected to further experimental processing as described below.

Experiment 1002 was carried out to determine the effect of temperature on the process being studied in comparison to AP plasma treatment. Accordingly, after the initial hydrolysis process 1022, the sample portion 1032 was then subjected to an oven treatment at block 1042. Specifically, the sample portion 1032 was placed in a laboratory oven and baked at 100° C. and ambient pressure for 20 minutes, yielding an oven-treated sample portion 1044. Experiment 1004 was continued under comparable thermal and temporal conditions by subjecting the sample portion 1032 to an AP plasma treatment 1052 for 20 minutes, yielding a plasma-treated sample portion 1054. The AP plasma treatment 1052 was implemented by utilizing an apparatus similar to the apparatus 500 described above and illustrated in FIG. 5. The working gases employed to generate and maintain the plasma were oxygen (O₂), helium (He) and neon (Ne). Experiment 1006 served as a control and therefore the sample portion 1036 was not subjected to any type of treatment other than hydrolysis.

Subsequently, in each of experimental pathways 1002, 1004 and 1006, the oven-treated sample portion 1044, plasma-treated sample portion 1054, and untreated control sample portion 1036 were respectively subjected to a second conventional, relatively concentrated acid hydrolysis process, at block 1062. Each hydrolysis process 1062 entailed placing the oven-treated sample portion 1044, plasma-treated sample portion 1054, or untreated control sample portion 1036 in a respective glass container containing a 19 mL mixture of 1.0-M H₂SO₄ and water. An identical container was employed for each of the oven-treated sample portion 1044, plasma-treated sample portion 1054, and untreated control sample portion 1036. For each hydrolysis process 1062, the mixture, including the acid, water, and the oven-treated sample portion 1044, plasma-treated sample portion 1054, or untreated control sample portion 1036, was heated at 100° C. for 75 minutes. Thereafter, at block 1064, the respective hydrolyzate sugar solutions were removed from each of the oven-treated sample portion 1044, plasma-treated sample portion 1054, and untreated control sample portion 1036 for analysis.

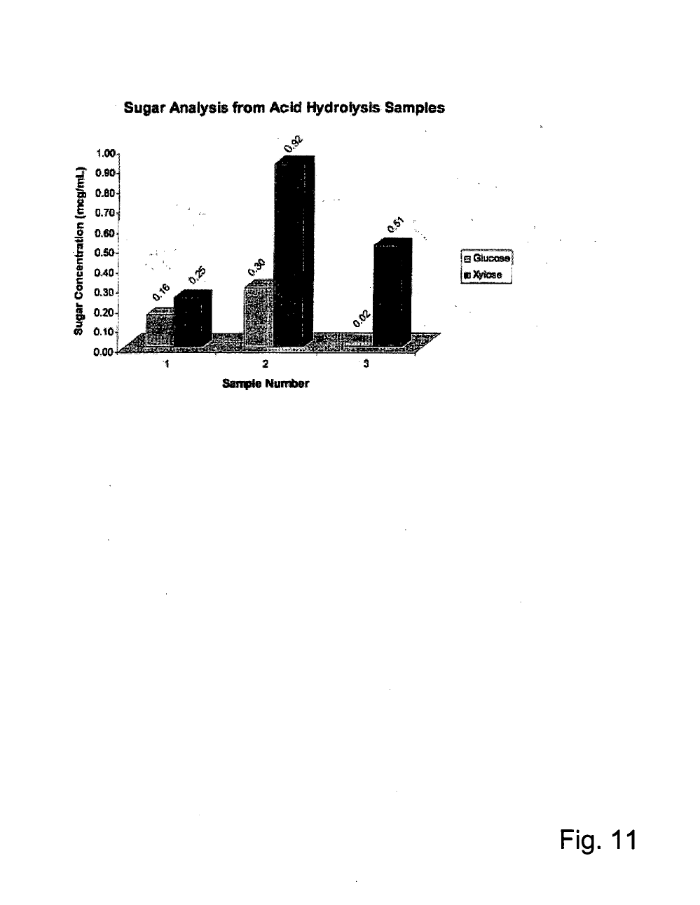

The six sugar solutions (derived from Sample #1, Sample #2, and Sample #3 and the two hydrolysis stages 1022 and 1062) were analyzed by an employing HPLC (high-performance liquid chromatography) technique, with particular emphasis on measuring the glucose and xylose concentrations of the sugar solutions. FIG. 11 illustrates data acquired from this analysis. In FIG. 11, the ordinate values represent sugar concentration (in μg/mL solution), and three pairs of bars are presented along the abscissa. The first pair corresponds to experimental pathway 1002 (oven treatment 1042 in FIG. 10), the second pair corresponds to experimental pathway 1004 (AP plasma treatment 1052 in FIG. 10), and the third pair corresponds to experimental pathway 1006 (control). For each pair, the left bar depicts glucose concentration and the right bar depicts xylose concentration. It will be noted that data obtained from the first-stage hydrolysis process 1022 (FIG. 10) was omitted due to the identical nature of each sample resulting from that stage. It was determined that sugar concentrations in liquid obtained from the second-stage hydrolysis process 1062 (FIG. 10) would adequately provide good indications of the effect each experiment 1002, 1004 and 1006 had on the maple sawdust Samples #1, #2 and #3.

It can be seen from FIG. 11 that Sample #2, the plasma-treated sample, produced significantly higher concentrations of both glucose and xylose as compared to that of Sample #1 (oven-treated sample) and Sample #3 (control sample). The AP treatment showed improvement in xylose and glucose concentrations of approximately 80% ((0.92-0.51)/0.51) and 88% ((0.30-0.16)/0.16), respectively, when compared to the closest data of the other two experiments. In view of the foregoing, FIGS. 10 and 11 demonstrate that AP plasma treatment of maple sawdust provides an improvement over current practices of acid hydrolysis treatment.

### EXAMPLE 2

This experiment utilized the reduction of potassium permanganate (KMnO₄) with glucose as a purple-pink indicator in conjunction with acid hydrolysis of pure cellulose. Deep purple-colored permanganate (MnO₄⁻) can be reduced to the faintly pink Mn⁺² cation in an acidic solution, where Mn is in a +2 oxidation state, as represented by the following stoichiometric expression:
MnO₄⁻+8H⁺+5e⁻→Mn⁺²+4H₂O.

The permanganate reacts with the glucose in solution, so as the process of acid hydrolysis is breaking down the cellulose into glucose, the permanganate is consumed, thus precipitating the loss of the purple or pink color over time. The amount of time required for the solution to lose its color indicates how effective the acid hydrolysis process is at breaking down the cellulose. Therefore, the effectiveness of AP plasma treatment as a precursor to acid hydrolysis will be indicated in the case where the permanganate/glucose reactions of plasma-treated samples run to completion faster than those of untreated cellulose.

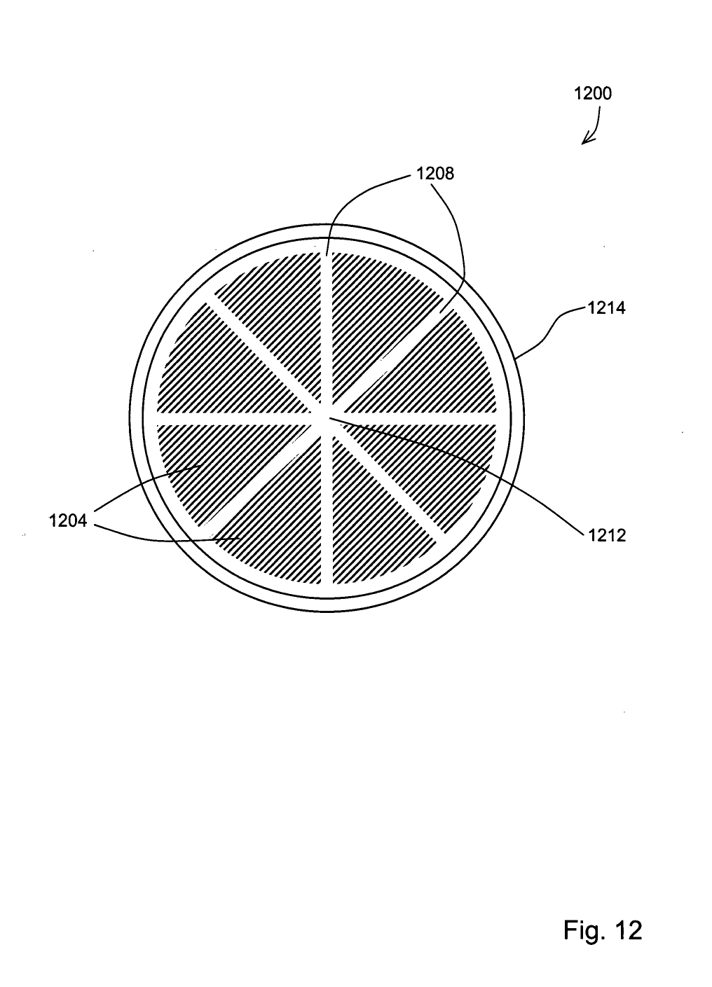

In this experiment, two plastic wafer holders were employed as containers and configured to hold eight individual samples of pure cellulose such that each sample was isolated from the other seven samples. FIG. 12 is a top plan view of one of the containers 1200. The container 1200 includes eight individual samples 1204 physically separated by gaps 1208 that radially extend from the center 1212 of the container 1200 to its peripheral region 1214, at equal angles to each other, such that the samples 1204 have the appearance of pie slices in FIG. 12. Sixteen 0.25-g samples of pure cellulose were prepared for treatment by an AP plasma apparatus, similar to the apparatus 500 described above and illustrated in FIG. 5, by placing each sample 1204 in the container 1200 and separated from the other samples 1204 in the manner illustrated in FIG. 12 Two containers 1200 were employed, each holding eight samples 1204. Four more 0.25-g samples of pure cellulose were prepared as controls for the experiment.

The first container 1200 of eight samples 1204 was placed into the AP plasma apparatus, which was then closed off. The background gas for the plasma was water-saturated helium. Specifically, a He gas line routed to the AP plasma apparatus was opened and allowed to bubble (percolate) through deionized (DI) water to produce a constant He/H₂O vapor gas flow. The gas lines and chamber of the AP plasma apparatus were allowed to purge for five to eight minutes. Once the system had purged, the voltage source of the AP plasma apparatus was engaged, thereby producing a pink-purple plasma. The samples 1204 were treated by the plasma generated in the AP plasma apparatus for five minutes. The container 1200 was then removed from the chamber and four of the eight samples 1204 were transferred into four other individual containers for subsequent hydrolysis. The AP plasma apparatus was prepared again in the same way as before, and the four remaining samples were treated for five more minutes, and thus for a total of ten minutes. This procedure was performed again for another eight samples, four of which were treated for twenty minutes and the other four for thirty minutes.

All plasma-treated samples were then subjected to dilute-acid hydrolysis, employing 1M H₂SO₄ (diluted from 18M H₂SO₄). After hydrolysis of the samples was completed, the effectiveness of the AP plasma treatment as an enhancement to the hydrolysis was analyzed according to the following procedure. Each 0.25-g sample was placed in a 100-mL round-bottom flask and 10 mL of tap water was added. The flask was then held in a boiling water bath (100° C.) by a ring stand, and a magnetic stir bar was operated to ensure proper mixing throughout the experiment. Five mL of 1M H₂SO₄ was added, followed by 2 mL of KMnO₄ (0.4 g_{KMnO4}/L_{H20} concentration). As soon as the permanganate was added, a timer was started. The reaction was allowed to run until the solution lost all of the pink color, at which point the timer was stopped. The flask and stir bar were thoroughly washed and the experiment was then repeated for the remaining samples.

The TABLE below tabulates the reaction times for each sample (20 total) with H₂SO₄ ans KMnO₄. The row of the TABLE providing the data for zero-minute treatment times corresponds to the untreated control samples. There appeared to be some degree of variation with the reaction times, which was determined to be due to the error involved in “eyeballing” when exactly the solutions lose all their color. However, the TABLE evidences that the times required for each reaction to run to completion were consistently shorter for all plasma-treated samples when compared to the untreated samples.

TABLE

| Treatment Time | | 1^{st} Run | 2^{nd} Run | 3^{rd} Run | 4^{th} Run |
|---|---|---|---|---|---|
| 0 | Min | 1:43 | 1:35 | 1:32 | 1:22 |
| 5 | min | 0:50 | 1:04 | 1:15 | 0:56 |
| 10 | min | 1:12 | 1:15 | 1:17 | 1:03 |
| 20 | min | 1:01 | 0:57 | 0:49 | 0:49 |
| 30 | min | 0:54 | 0:43 | 0:51 | 0:50 |

The consistency of the shorter reaction times for treated samples indicates that the AP plasma treatment of the cellulose facilitates the hydrolysis of cellulose into glucose.

### EXAMPLE 3

This experiment was conducted to determine whether AP plasma treatment itself was capable of producing glucose from cellulose. Three samples of pure cellulose were provided, weighing 225.1 mg, 231 mg and 226.9 mg, respectively. Each sample was dried in a vacuum oven under 30 mm Hg of pressure at a temperature of 110° C. for two and one half hours. This allowed for full evaporation of the water in the cellulose, using knowledge gained from previous experiments that determined the time to dry 200 mg of cellulose was approximately one hour. After the drying cycle, the dry weights of the three samples were measured to be 203, 210.9 and 206.9 mg, respectively. Each sample was then treated by AP plasma by placing the sample in an AP plasma apparatus, similar to the apparatus 500 described above and illustrated in FIG. 5, and exposing the sample to a plasma generated by the AP plasma apparatus. The AP plasma apparatus was operated at a voltage of 5 kV and a current of 50 mA. Alumina dielectrics were employed between the two electrodes of the apparatus, and were spaced apart by a one-inch gap. The plasma medium was water vapor in helium, similar to that described above in connection with EXAMPLE 2. The three samples were plasma-treated for ten, twenty and thirty minutes, respectively.

During the plasma treatments, the three samples were analyzed with an optical spectrometer that was integrated with the AP plasma apparatus. Peak emissions were observed at 309.56-314.92 (molecular peak), 336.48, 353.64, 357.69, 380.43, 389.13, 587.84, 656.36, 667.90, 706.72, and 728.22 nm.

After the plasma treatments, the three samples were then placed into respective 100-mL beakers with 50 mL of DI water. Using a hotplate and magnetic stir bars, the three samples were boiled and stirred in the beakers for one hour. This step was completed to ensure that if glucose was produced by the AP plasma treatment, then the glucose would dissolve in the water leaving behind only the cellulose not converted by the plasma treatment. After the boiling/stirring cycle, the solutions of the three samples were each separated from the unconverted residual material by employing a syringe with a 1-micron filter paper.

After separation, the three samples were placed into the vacuum oven under the same conditions described above prior to the AP plasma treatment. After two hours of drying, the dry weights of the treated samples were found to be 188.3, 193.4 and 190.0 mg for the 10-min, 20-min and 30-min treatments, respectively. These weights represent the amount of cellulose not converted by the AP plasma treatment for each sample. By taking the original, pre-treatment dry weights and subtracting the post-treatment dry weights for each sample, it was determined that the amounts of glucose produced as a result of the AP plasma treatment were 14.7, 17.6 and 15.2 mg, respectively. This demonstrates 7.2%, 8.3% and 7.4% conversion of cellulose to glucose for the 10-min, 20-min and 30-min treatments, respectively. Therefore, the foregoing data indicate that the AP plasma treatment itself is capable of releasing glucose from cellulose, without the use of conventional techniques such as hydrolysis, and further demonstrates the ability of AP plasma treatment to promote conversion of cellulose and cellulose-containing materials to alcohol.

The foregoing results of the EXAMPLES are significant as they relate to the long felt need related to economic and ecological aspects of this invention. Fewer steps are required, and expensive acids, high temperatures, high pressures, and costly enzymes are bypassed by providing a direct pathway to the sugars through plasma-enhanced soft degradation as taught in the present disclosure.

While the foregoing disclosure has focused primarily on the treatment of cellulose, the invention may also be applicable to the treatment of starch. As readily appreciated by persons skilled in the art, starch is a water-insoluble, complex carbohydrate containing around 2500 glucose monomer units. In general, starches have the formula (C₆H₁₀O₅)ₙ, where “n” denotes the total number of glucose monomer units. More specifically, starch is a combination of the two polysaccharides amylose and amylopectin. Amylose constitutes a straight chain of glucose units joined to one another by α-1,4 linkages. Amylopectin, on the other hand, includes branches, with an α-1,6 linkage every 24-30 glucose units. That is, starch forms clusters of linked linear polymers, where the α-1,4 linked chains form columns of glucose units which branch regularly at the α-1,6 links. As previously noted, a starch molecule as a result has a coiled conformation unlike a straight-chain cellulose molecule. Starches can be digested by hydrolysis into simpler saccharide units. The hydrolysis may be catalyzed by enzymes known as amylases, which break the glycosidic bonds between the α-glucose components of the starch polysaccharide molecule.

The foregoing description of implementations has been presented for purposes of illustration and description. It is not exhaustive and does not limit the claimed inventions to the precise form disclosed. Modifications and variations are possible in light of the above description or may be acquired from practicing the invention. The claims and their equivalents define the scope of the invention.

What is claimed is:

1. A method for treating a cellulosic material, comprising subjecting the cellulosic material to an atmospheric-pressure plasma to produce a plasma-treated cellulosic material.

2. The method of claim 1, wherein subjecting includes introducing the cellulosic material to a plasma-generating apparatus, and operating the apparatus to generate the atmospheric-pressure plasma from a plasma medium present in the apparatus.

3. The method of claim 2, wherein operating the apparatus includes operating a plasma-generating apparatus selected from the group consisting of a dielectric barrier discharge apparatus, a microplasma-generating apparatus, a plasma-generating apparatus including a parallel-plate configuration, a plasma-generating apparatus including a drop-tube configuration, a plasma-generating apparatus including a fluidized-bed configuration, a plasma-generating apparatus including a liquid-bath configuration, and a plasma-generating apparatus including a plasma-jet configuration.

4. The method of claim 2, wherein the atmospheric-pressure plasma is generated from a plasma medium selected from the group consisting of air, oxygen, hydrogen, helium, water-saturated helium, neon, argon, and combinations of two or more of the foregoing.

5. The method of claim 1, wherein the plasma-treated cellulosic material includes one or more degraded species.

6. The method of claim 5, wherein the one or more degraded species include one or more sugars.

7. The method of claim 6, wherein the one or more sugars include glucose.

8. The method of claim 5, further including separating one or more of the degraded species from a remaining portion of the plasma-treated cellulosic material.

9. The method of claim 1, further including fermenting one or more components of the plasma-treated cellulosic material.

10. The method of claim 9, wherein fermenting includes allowing the one or more components to interact with one or more fermentative enzymes at a temperature at which the one or more enzymes exhibit fermenting activity.

11. The method of claim 10, wherein the one or more enzymes include microorganisms.

12. The method of claim 10, wherein the one or more enzymes include yeast.

13. The method of claim 9, wherein fermenting produces a fermentation product including an organic compound.

14. The method of claim 13, wherein the organic compound includes an alcohol.

15. The method of claim 14, wherein the alcohol includes ethanol.

16. The method of claim 1, further including subjecting the cellulosic material to an additional treatment process selected from the group consisting of comminution, steam explosion, hydrothermolysis, acid addition, alkali addition, solvent addition, ammonia addition, wet oxidation, ammonia fiber/freeze explosion (AFEX), ammonia recycled percolation (ARP), hydrolysis, acid-assisted hydrolysis, enzyme-assisted hydrolysis, and combinations of two or more of the foregoing.

17. A sugar produced according to the method of claim 1.

18. The sugar of claim 17, wherein the sugar includes glucose.

19. A fermentation product produced by fermenting one or more components of a cellulosic material treated according to the method of claim 1.

20. The fermentation product of claim 19, wherein the fermentation product includes an organic compound.

21. The fermentation product of claim 20, wherein the organic compound includes ethanol.

22. A method for treating a cellulosic material, comprising:
subjecting the cellulosic material to an atmospheric-pressure plasma to produce a plasma-treated cellulosic material; and
subjecting one or more components of the plasma-treated cellulosic material to a degradation process to produce a degradation-processed cellulosic material.

23. The method of claim 22, wherein subjecting the cellulosic material to the atmospheric-pressure plasma includes introducing the cellulosic material to a plasma-generating apparatus, and operating the apparatus to generate the atmospheric-pressure plasma from a plasma medium present in the apparatus.

24. The method of claim 22, wherein the plasma-treated cellulosic material includes one or more degraded species.

25. The method of claim 24, further including separating one or more of the degraded species from a remaining portion of the plasma-treated cellulosic material, wherein the remaining portion includes the one or more components of the plasma-treated cellulosic material subjected to the degradation process.

26. The method of claim 24, further including fermenting one or more components of the degraded species.

27. The method of claim 26, wherein fermenting produces a fermentation product including ethanol.

28. The method of claim 22, wherein subjecting the one or more components of the plasma-treated cellulosic material to the degradation process includes performing hydrolysis.

29. The method of claim 28, wherein performing hydrolysis includes performing acid hydrolysis.

30. The method of claim 29, wherein performing acid hydrolysis includes combining the one or more components of the plasma-treated cellulosic material with a liquid and an acidic component, at a temperature sufficient for the acidic component to catalyze hydrolysis.

31. The method of claim 30, wherein the acidic component is selected from the group consisting of mineral acids, acidic salts and combinations of the foregoing.

32. The method of claim 28, wherein performing hydrolysis includes performing enzymatic hydrolysis.

33. The method of claim 32, wherein performing enzymatic hydrolysis includes combining the one or more components of the plasma-treated cellulosic material with a liquid and an enzyme, at a temperature sufficient for the enzyme to catalyze hydrolysis.

34. The method of claim 33, wherein the enzyme is selected from the group consisting of cellulases, hemicellulases, and combinations of the foregoing.

35. The method of claim 22, wherein the degradation-processed cellulosic material includes one or more monomeric sugars.

36. The method of claim 22, further including fermenting one or more components of the degradation-processed cellulosic material.

37. The method of claim 36, wherein fermenting produces a fermentation product including ethanol.

38. The method of claim 22, further including:
separating a first portion of the plasma-treated cellulosic material from a second portion of the plasma-treated cellulosic material, wherein the second portion includes the one or more components of the plasma-treated cellulosic material subjected to the degradation process;
fermenting one or more components of the first portion to produce a first fermentation product; and
fermenting one or more components of the degradation-processed cellulosic material to produce a second fermentation product.

39. The method of claim 38, wherein at least one of the first and second fermentation products includes ethanol.

40. The method of claim 38, wherein the one or more components of the first portion of the plasma-treated cellulosic material are fermented together with the one or more components of the degradation-processed cellulosic material.

41. The method of claim 22, further including subjecting the cellulosic material to an additional treatment process selected from the group consisting of comminution, steam explosion, hydrothermolysis, acid addition, alkali addition, solvent addition, ammonia fiber/freeze explosion (AFEX), ammonia recycled percolation (ARP), and combinations of two or more of the foregoing.

42. A method for treating a cellulosic material, comprising:
subjecting the cellulosic material to a first degradation process to produce a first degradation-processed cellulosic material;
subjecting one or more components of the first degradation-processed cellulosic material to an atmospheric-pressure plasma to produce a plasma-treated cellulosic material; and
subjecting one or more components of the plasma-treated cellulosic material to a second degradation process to produce a second degradation-processed cellulosic material.

43. The method of claim 42, wherein subjecting the one or more components of the first degradation-processed cellulosic material to the atmospheric-pressure plasma includes introducing the one or more components of the first degradation-processed cellulosic material to a plasma-generating apparatus, and operating the apparatus to generate the atmospheric-pressure plasma from a plasma medium present in the apparatus.

44. The method of claim 42, wherein at least one of the first and second degradation processes includes hydrolysis.

45. The method of claim 42, wherein subjecting the cellulosic material to the first degradation process includes performing a first hydrolysis, and subjecting the one or more components of the plasma-treated cellulosic material to the second degradation process includes performing a second hydrolysis.

46. The method of claim 45, wherein performing the first hydrolysis includes performing a first acid hydrolysis and performing the second hydrolysis includes performing a second acid hydrolysis.

47. The method of claim 46, wherein the first acid hydrolysis is performed at a lower acidic concentration than the second acid hydrolysis.

48. The method of claim 45, wherein performing the first hydrolysis includes performing an acid hydrolysis and performing the second hydrolysis includes performing an enzymatic hydrolysis.

49. The method of claim 45, wherein performing the first hydrolysis includes performing an enzymatic hydrolysis and performing the second hydrolysis includes performing an acid hydrolysis.

50. The method of claim 42, wherein the first degradation-processed cellulosic material includes one or more monomeric sugars.

51. The method of claim 50, further including fermenting one or more of the monomeric sugars.

52. The method of claim 51, wherein fermenting produces a fermentation product including ethanol.

53. The method of claim 42, wherein the plasma-treated cellulosic material includes one or more monomeric sugars.

54. The method of claim 53, further including fermenting one or more of the monomeric sugars.

55. The method of claim 54, wherein fermenting produces a fermentation product including ethanol.

56. The method of claim 42, wherein the second degradation-processed cellulosic material includes one or more monomeric sugars.

57. The method of claim 56, further including fermenting one or more of the monomeric sugars.

58. The method of claim 57, wherein fermenting produces a fermentation product including ethanol.

59. The method of claim 42, wherein the first degradation-processed cellulosic material includes one or more monomeric sugars and the plasma-treated cellulosic material includes one or more monomeric sugars, and the method further includes fermenting the one or more monomeric sugars of the first degradation-processed cellulosic material, and fermenting the one or more monomeric sugars of the plasma-treated cellulosic material.

60. The method of claim 42, wherein the first degradation-processed cellulosic material includes one or more monomeric sugars, the plasma-treated cellulosic material includes one or more monomeric sugars, and the second degradation-processed cellulosic material includes one or more monomeric sugars, and the method further includes fermenting the one or more monomeric sugars of the first degradation-processed cellulosic material, fermenting the one or more monomeric sugars of the plasma-treated cellulosic material, and fermenting the one or more monomeric sugars of the second degradation-processed cellulosic material.

61. The method of claim 42, wherein the plasma-treated cellulosic material includes one or more monomeric sugars and the second degradation-processed cellulosic material includes one or more monomeric sugars, and the method further includes fermenting the one or more monomeric sugars of the plasma-treated cellulosic material, and fermenting the one or more monomeric sugars of the second degradation-processed cellulosic material.

62. The method of claim 61, wherein the one or more monomeric sugars of the plasma-treated cellulosic material are fermented together with the one or more monomeric sugars of the second degradation-processed cellulosic material.

63. The method of claim 42, wherein at least one of the first degradation-processed cellulosic material, the plasma-treated cellulosic material, and the second degradation-processed cellulosic material includes one or more monomeric sugars, and the method further includes fermenting the one or more monomeric sugars to produce a fermentation product including ethanol.

64. The method of claim 42, further including subjecting the cellulosic material to an additional treatment process selected from the group consisting of comminution, steam explosion, hydrothermolysis, acid addition, alkali addition, solvent addition, ammonia fiber/freeze explosion (AFEX), ammonia recycled percolation (ARP), and combinations of two or more of the foregoing.

65. A method for treating a cellulosic material, comprising:
subjecting the cellulosic material to a pretreatment process;
subjecting the cellulosic material to an atmospheric-pressure plasma; and
subjecting the cellulosic material to a degradation process.

66. The method of claim 65, wherein subjecting the cellulosic material to the atmospheric-pressure plasma includes introducing the cellulosic material to a plasma-generating apparatus, and operating the apparatus to generate the atmospheric-pressure plasma from a plasma medium present in the apparatus.

67. The method of claim 65, wherein subjecting the cellulosic material to the pretreatment process produces a pretreated cellulosic material, and subjecting the cellulosic material to the atmospheric-pressure plasma occurs after the pretreatment process and includes subjecting one or more components of the pretreated cellulosic material to the atmospheric-pressure plasma.

68. The method of claim 65, wherein subjecting the cellulosic material to the atmospheric-pressure plasma produces a plasma-treated cellulosic material, and the pretreatment process occurs after producing the plasma-treated cellulosic material and includes subjecting one or more components of the plasma-treated cellulosic material to the pretreatment process.

69. The method of claim 65, wherein:
subjecting the cellulosic material to the atmospheric-pressure plasma includes subjecting the cellulosic material to at least a first plasma treatment and a subsequent second plasma treatment, the pretreatment process occurring between the first and second plasma treatments;
subjecting the cellulosic material to the first plasma treatment produces a first plasma-treated cellulosic material, and subjecting the cellulosic material to the pretreatment process includes subjecting one or more components of the first plasma-treated cellulosic material to the pretreatment process to produce a pretreated cellulosic material;
subjecting the cellulosic material to the second plasma treatment includes subjecting one or more components of the pretreated cellulosic material to the second plasma treatment to produce a second plasma-treated cellulosic material; and
subjecting the cellulosic material to the degradation process includes subjecting one or more components of the second plasma-treated cellulosic material to the degradation process.

70. The method of claim 65, wherein the pretreatment process is selected from the group consisting of comminution, steam explosion, hydrothermolysis, acid addition, alkali addition, solvent addition, ammonia fiber/freeze explosion (AFEX), ammonia recycled percolation (ARP), and combinations of two or more of the foregoing.

71. The method of claim 65, wherein subjecting the cellulosic material to an atmospheric-pressure plasma produces a plasma-treated cellulosic material, and the method further includes fermenting one or more components of the plasma-treated cellulosic material.

72. The method of claim 71, wherein fermenting produces a fermentation product including ethanol.

73. The method of claim 65, wherein subjecting the cellulosic material to a degradation process produces a degradation-processed cellulosic material, and the method further includes fermenting one or more components of the degradation-processed cellulosic material.

74. The method of claim 73, wherein fermenting produces a fermentation product including ethanol.

75. The method of claim 65, wherein subjecting the cellulosic material to an atmospheric-pressure plasma produces a plasma-treated cellulosic material and subjecting the cellulosic material to a degradation process produces a degradation-processed cellulosic material, and the method further includes fermenting one or more components of the plasma-treated cellulosic material to produce a first fermentation product and fermenting one or more components of the degradation-processed cellulosic material to produce a second fermentation product.

76. The method of claim 75, wherein at least one of the first and second fermentation products includes ethanol.

77. A cellulosic material treatment apparatus, comprising:
a first electrode;
a second electrode;
means for providing a cellulosic material between the first and second electrodes; and
means for subjecting the cellulosic material to an atmospheric-pressure plasma between the first and second electrodes.

78. The cellulosic material treatment apparatus of claim 77, wherein the first electrode and the second electrode comprise an electrode arrangement selected from the group consisting of a first plate and a second plate spaced from the first plate, a first annular electrode and a second annular electrode coaxially disposed about a container, a first electrode disposed in a container and a second electrode coaxially disposed about the container, a first electrode and a second electrode spaced from the first electrode in a container, and an elongated electrode and a hollow electrode coaxially disposed about the elongated electrode.

79. The cellulosic material treatment apparatus of claim 77, wherein the providing means is selected from the group consisting of a planar surface, a container, an enclosure having an opening, a flow-through conduit having an inlet and an outlet, a liquid-bath container, and a plasma-jet enclosure.

80. The cellulosic material treatment apparatus of claim 77, wherein the subjecting means includes a plasma-generating apparatus selected from the group consisting of a dielectric barrier discharge apparatus, a microplasma-generating apparatus, a plasma-generating apparatus including a parallel-plate configuration, a plasma-generating apparatus including a drop-tube configuration, a plasma-generating apparatus including a fluidized-bed configuration, a plasma-generating apparatus including a liquid-bath configuration, and a plasma-generating apparatus including a plasma-jet configuration.

81. The cellulosic material treatment apparatus of claim 77, wherein the subjecting means includes a plasma-generating apparatus utilizing a plasma medium selected from the group consisting of air, oxygen, hydrogen, helium, water-saturated helium, neon, argon, and combinations of two or more of the foregoing.
